(19) United States
(12) Patent Application Publication
(10) Pub. No.: US 2024/0317774 A1
(43) Pub. Date: Sep. 26, 2024

(54) Pharmaceutical Compound

(71) Applicant: GLAXOSMITHKLINE INTELLECTUAL PROPERTY DEVELOPMENT LIMITED, Stevenage (GB)

(72) Inventors: M Pilar Manzano-Chinchon, Tres Cantos (ES); Margarita Puente-Felipe, Tres Cantos (ES); Kate C Dennis, Stevenage (GB); Jose Maria Bueno Calderon, Tres Cantos (ES)

(21) Appl. No.: 18/569,648

(22) PCT Filed: Jun. 15, 2022
(86) PCT No.: PCT/EP2022/066391

§ 371 Date: Dec. 13, 2023

(30) Foreign Application Priority Data
Jun. 15, 2021 (EP) 21382532.6

(51) Int. Cl. C07D 495/04; A61K 31/4365; A61K 45/06; A61P 33/06

(52) U.S. Cl. CPC C07D 495/04; A61K 31/4365; A61K 45/06; A61P 33/06

(57) ABSTRACT

The present application relates to compounds of Formula (I) or a pharmaceutically acceptable salt thereof, compositions thereof, and their use in the treatment and/or prophylaxis of systemic infections, such as the treatment and/or prophylaxis of systemic parasitic and fungal infections, such as malaria, in particular infection by Plasmodium falciparum.

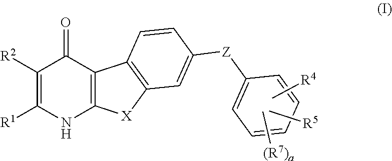

### FIELD OF THE INVENTION

The present application relates to compounds and pharmaceutically acceptable salts and tautomers thereof, compositions thereof, formulations thereof, and their use in the treatment and/or prophylaxis of systemic infections, such as the treatment and/or prophylaxis of systemic parasitic and fungal infections, such as malaria, in particular infection by Plasmodium falciparum.

### BACKGROUND TO THE INVENTION

Parasitic infections are responsible for a wide variety of diseases of medical and veterinary importance, for example malaria in humans and coccidiosis in birds, fish, and mammals. Many of the diseases are life-threatening to the host and cause considerable economic loss in animal husbandry.

Malaria is a disease caused by protozoan parasites of the genus Plasmodium that infect and destroy red blood cells, leading to fever, severe anaemia, cerebral malaria, and if untreated, death. There are five species of Plasmodium parasite: falciparum, vivax, ovale, malariae, and knowlesi. Plasmodium falciparum is the most virulent. In 2019, there were an estimated 229 million people infected with malaria in 87 malaria endemic countries, and malarial disease was responsible for an estimated 409,000 deaths (World malaria report 2020: 20 years of global progress and challenges. Geneva: World Health Organisation; 2020, Switzerland).

There is currently no vaccine against malaria that is effective in all patient populations. Effective oral preparations for the prophylaxis and treatment of malaria are known in the art. For example, known treatments/prophylaxis include doxycycline, mefloquine, chloroquine, primaquine, quinidine, artesunate, and atovaquone. However, the treatment/prophylaxis of malaria generally requires the patient to take a tablet daily. For example, prophylactic treatment with a combination of atovaquone and proguanil hydrochloride (sold as Malarone®) requires the patient to take a daily tablet while in the malarial area, with the treatment starting 24-48 hours before entering the malarial area, and continuing for 7 days after leaving the malarial area (Malarone 250 mg/100 mg film-coated tablets, summary of product characteristics).

Poor patient compliance/adherence to these strict treatment regimens often leads to failure of the treatment/prophylaxis, which leads to increased risk of the patient contracting malaria. Poor patient compliance (e.g., missed doses or untimely dosing) may also lead to sub-therapeutic drug concentrations being obtained in the patient. This puts the patient at risk of contracting malaria and allows for drug resistant malaria to develop. Conventional therapies also struggle to deal with seasonal malaria in endemic areas.

There is therefore a need to develop improved compounds for the treatment/prophylaxis of malaria that allow for a reduction in dosing and improved ease of adherence of treatment.

### SUMMARY OF THE INVENTION

In a first aspect of the invention, a compound according to Formula (I)

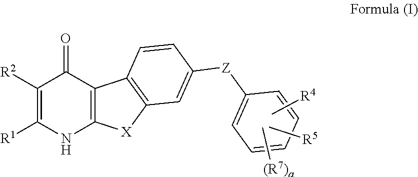

or a pharmaceutically acceptable salt thereof, wherein:
R¹ is C₁₋₅ alkyl, C₁₋₅ hydroxyalkyl or C₁-C₅ haloalkyl;
R² is hydrogen or halogen;
X is S, SO, or SO₂;
Z is a single bond, —O—, —C═C—, —C═C—CH₂—NR³—CH₂—, or —(CH₂)ₘ—, where m is 1, 2 or 3, and where R³ is hydrogen or a C₁₋₅ alkyl;
R⁴ is hydrogen, halogen, —CH₂F, —CHF₂, —CF₃, —OCH₂F, —OCHF₂, or —OCF₃;
R⁵ is —CF₃, —OCF₃, —(CH₂)_{q}—OH, where q is 1, 2, 3, or 4, or R⁵ is represented by the following group:

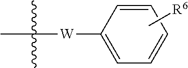

where [symbol] indicates a binding site to the phenyl ring and wherein W is —O—, or —CH₂—NR^{b}—Y—CH₂—, where R^{b} is hydrogen or C₁₋₅ alkyl, and Y is a single bond or Y is represented by the following group:

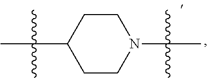 [SMILES: *C1CCN(*)CC1]

where [symbol] indicates a binding site to the amine and [symbol] indicates a binding site to the methylene group;
R⁶ is —CF₃ or —OCF₃;
R⁷ is halogen, —CHF₂, —CH₂F, or —CF₃, and a is 0, 1, 2, or 3.

In a second aspect of the invention, there is provided a crystalline form of 3-chloro-7-(2-fluoro-4-(trifluoromethyl)phenyl)-2-methylbenzo[4,5] thieno[2,3-b]pyridin-4(1H)-one characterised by an X-ray powder diffraction (XRPD) pattern comprising at least three diffraction angles, when measured using Cu K_{α} radiation, selected from the group consisting of either: (i) about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ; or (ii) about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ.

In a third aspect of the invention, there is provided a compound of Formula (I) or a pharmaceutically acceptable salt thereof for use in therapy.

In a fourth aspect of the invention, there is provided a pharmaceutical composition comprising (a) a compound of Formula (I) or pharmaceutically acceptable salt thereof; and (b) a pharmaceutically acceptable excipient.

In a fifth aspect of the invention, there is provided a compound of Formula (I) or a pharmaceutically acceptable salt thereof for use in the treatment and/or prophylaxis of a parasitic protozoal infection.

In a sixth aspect of the invention, there is provided a method of treating a parasitic protozoal infection in a patient in need thereof, comprising administering to the patient a therapeutically effective amount of the compound of Formula (I) or pharmaceutically acceptable salt thereof or a pharmaceutical composition comprising the compound of Formula (I) or pharmaceutically acceptable salt thereof.

In a seventh aspect of the invention, there is provided the use of a compound of Formula (I) or pharmaceutically acceptable salt thereof in the manufacture of a medicament for use in the treatment and/or prophylaxis of a parasitic protozoal infection.

In an eighth aspect of the invention, there is provided a combination of (a) a compound of Formula (I) or a pharmaceutically acceptable salt thereof; and (b) at least one other anti-malarial agent.

### BRIEF DESCRIPTION OF THE DRAWINGS

The invention is further described by reference to the accompanying drawings, which are non-limiting.

FIG. 1 shows an X-ray powder diffraction pattern of 3-chloro-7-(2-fluoro-4-(trifluoromethyl)phenyl)-2-methylbenzo[4,5] thieno[2,3-b]pyridin-4(1H)-one. The crystal form characterised in this figure is referred to herein as Form 1.

FIG. 2 shows another X-ray powder diffraction pattern of 3-chloro-7-(2-fluoro-4-(trifluoromethyl)phenyl)-2-methylbenzo[4,5] thieno[2,3-b]pyridin-4(1H)-one. The crystal form characterised in this figure is referred to herein as Form 2.

FIGS. 3-8 show pharmacokinetic data for six different formulations according to the invention.

### DETAILED DESCRIPTION OF THE EMBODIMENTS

### Statement of the Invention

As described above, in a first aspect of the invention, there is provided a compound according to Formula (I)

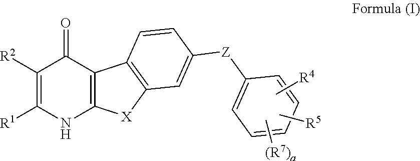

or a pharmaceutically acceptable salt or tautomer thereof, wherein:
R¹ is C₁₋₅ alkyl, C₁₋₅ hydroxyalkyl or C₁-C₅ haloalkyl;
R² is hydrogen or halogen;
X is S, SO, or SO₂;
Z is a single bond, —O—, —C═C—, —C═C—CH₂—NR³—CH₂—, or —(CH₂)ₘ—, wherein m is 1, 2 or 3, and wherein R³ is hydrogen or a C₁₋₅ alkyl;
R⁴ is hydrogen, halogen, —CH₂F, —CHF₂, —CF₃, —OCH₂F, —OCHF₂, or —OCF₃;
R⁵ is —CF₃, —OCF₃, —(CH₂)_{q}—OH, wherein q is 1, 2, 3, or 4, or R⁵ is represented by the following group:

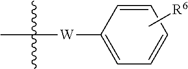

where [symbol] indicates a binding site to the phenyl ring and wherein W is —O—, or —CH₂—NR^{b}—Y—CH₂—, where R^{b} is hydrogen or C₁₋₅ alkyl, and Y is a single bond or Y is represented by the following group:

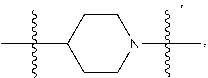 [SMILES: *C1CCN(*)CC1]

where [symbol] indicates a binding site to the amine and [symbol] indicates a binding site to the methylene group;
R⁶ is —CF₃ or —OCF₃;
R⁷ is halogen, —CHF₂, —CH₂F, or —CF₃; and
a is 0, 1, 2, or 3.

In some embodiments, the compound of the invention is defined according to Formula (Ia):

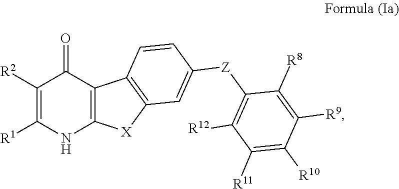

where R¹, R², X, and Z are as defined above for Formula (I),
R⁸ is hydrogen, halogen, —CHF₂, —CH₂F, or —CF₃;
R⁹ is hydrogen, —CF₃, or —OCF₃;
R¹⁰ is hydrogen, —CF₃, —OCF₃, C₁-C₄ alkyl-OH,

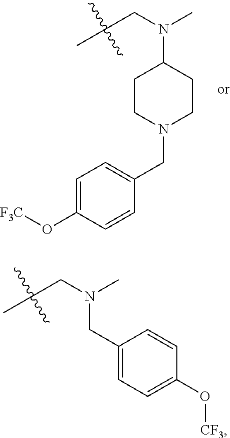 [SMILES: *CN(C)C1CCN(Cc2ccc(OC(F)(F)F)cc2)CC1 ; *CN(C)Cc1ccc(OC(F)(F)F)cc1]

where [symbol] indicates a binding site to the phenyl ring;
R¹¹ is hydrogen, —CHF₂, —CH₂F, or —CF₃; and
R¹² is hydrogen, halogen, —CHF₂, —CH₂F, —CF₃, or —OCF₃.

In some embodiments, R¹ is methyl. In other embodiments, R² is chloro. In still other embodiments, R¹ is methyl and R² is chloro.

In some embodiments, X is S. In other embodiments, X is S and R¹ is methyl. In still other embodiments, X is S and R² is chloro. In other embodiment, R¹ is methyl, R² is chloro, and X is S.

In some embodiments, Z is a single bond, wherein a single bond for Z is defined as no additional atoms between the two ring moieties but rather one single bond connecting the rings. In other embodiments, Z is a single bond and R¹ is methyl. In still other embodiments, Z is a single bond and R² is chloro. In other embodiments, Z is a single bond and X is S. In some embodiments, Z is a single bond, R¹ is methyl and R² is chloro. In other embodiments, Z is a single bond, R¹ is methyl and X is S. In still other embodiments, Z is a single bond, R² is chloro and X is S. In some embodiments, Z is a single bond, R¹ is methyl, R² is chloro, and X is S.

In some embodiments, the compound of the invention is defined according to Formula (Ia) and R¹ is methyl. In other embodiments, the compound of the invention is defined according to Formula (Ia) and R² is chloro. In still other embodiments, the compound of the invention is defined according to Formula (Ia), R¹ is methyl and R² is chloro. In some embodiments, the compound of the invention is defined according to Formula (Ia) and Z is a single bond, R¹ is methyl, R² is chloro, and X is S.

In some embodiments, the compound of Formula (I) or pharmaceutically acceptable salt thereof is:
3-chloro-7-(2-fluoro-4-(trifluoromethyl)phenyl)-2-methylbenzo[4,5] thieno[2,3-b]pyridin-4(1H)-one

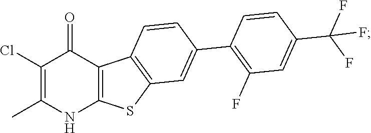 [SMILES: Cc1[nH]c2sc3cc(-c4ccc(C(F)(F)F)cc4F)ccc3c2c(=O)c1Cl]

sodium 3-chloro-7-[2-fluoro-4-(trifluoromethyl)phenyl]-2-methyl[1]benzothieno[2,3-b]pyridin-4-olate

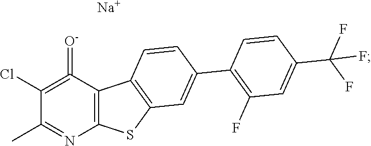

3-chloro-7-(2-fluoro-5-(trifluoromethyl)phenyl)-2-methylbenzo[4,5]thieno[2,3-b]pyridin-4(1H)-one

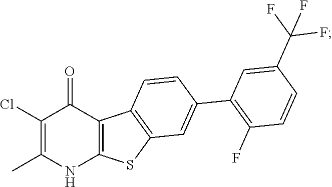 [SMILES: Cc1[nH]c2sc3cc(-c4cc(C(F)(F)F)ccc4F)ccc3c2c(=O)c1Cl]

3-chloro-2-methyl-7-(2-(trifluoromethoxy)phenyl)benzo[4,5]thieno[2,3-b]pyridin-4(1H)-one

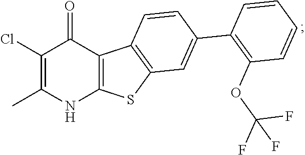 [SMILES: Cc1[nH]c2sc3cc(-c4ccccc4OC(F)(F)F)ccc3c2c(=O)c1Cl]

7-(2,4-bis(trifluoromethyl)phenyl)-3-chloro-2-methylbenzo[4,5]thieno[2,3-b]pyridin-4(1H)-one

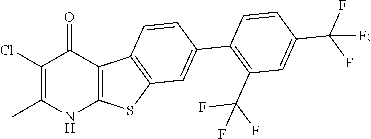 [SMILES: Cc1[nH]c2sc3cc(-c4ccc(C(F)(F)F)cc4C(F)(F)F)ccc3c2c(=O)c1Cl]

3-chloro-7-(4-(hydroxymethyl)phenyl)-2-methylbenzo[4,5]thieno[2,3-b]pyridin-4(1H)-one

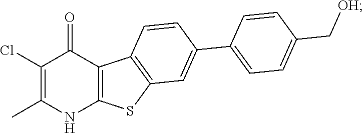 [SMILES: Cc1[nH]c2sc3cc(-c4ccc(CO)cc4)ccc3c2c(=O)c1Cl]

3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one

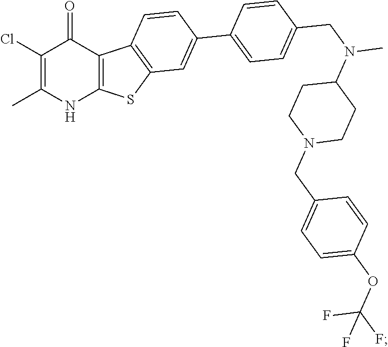 [SMILES: Cc1[nH]c2sc3cc(-c4ccc(CN(C)C5CCN(Cc6ccc(OC(F)(F)F)cc6)CC5)cc4)ccc3c2c(=O)c1Cl]

or
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one 2,2,2-trifluoroacetate

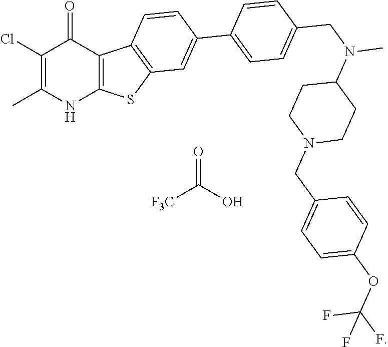 [SMILES: Cc1[nH]c2sc3cc(-c4ccc(CN(C)C5CCN(Cc6ccc(OC(F)(F)F)cc6)CC5)cc4)ccc3c2c(=O)c1Cl ; O=C(O)C(F)(F)F]

In some embodiments, the compound of Formula (I) is

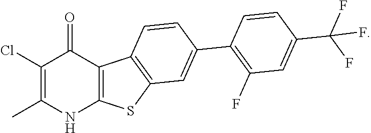 [SMILES: Cc1[nH]c2sc3cc(-c4ccc(C(F)(F)F)cc4F)ccc3c2c(=O)c1Cl]

In some embodiments, a compound which is

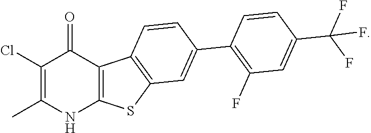 [SMILES: Cc1[nH]c2sc3cc(-c4ccc(C(F)(F)F)cc4F)ccc3c2c(=O)c1Cl]

In some embodiments, a compound which is

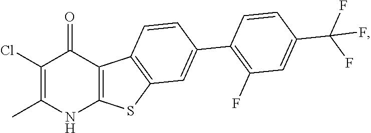 [SMILES: Cc1[nH]c2sc3cc(-c4ccc(C(F)(F)F)cc4F)ccc3c2c(=O)c1Cl]

or a pharmaceutically acceptable salt thereof.

In some aspects of the invention, a crystalline form of 3-chloro-7-(2-fluoro-4-(trifluoromethyl)phenyl)-2-methylbenzo[4,5] thieno[2,3-b]pyridin-4(1H)-one is provided characterised by an X-ray powder diffraction (XRPD) pattern comprising at least three diffraction angles, when measured using Cu K_{α} radiation, selected from the group consisting of about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least four diffraction angles, when measured using Cu K_{α} radiation, selected from the group consisting of about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least five diffraction angles, when measured using Cu K_{α} radiation, selected from the group consisting of about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least six diffraction angles, when measured using Cu K_{α} radiation, selected from the group consisting of about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least seven diffraction angles, when measured using Cu K_{α} radiation, selected from the group consisting of about 5.2±0.1, 8.6±0.1, 10.5, 12.5±0.1, 13.5±0.1, 15.6±0.1, 18.8±0.1, 19.7±0.1, 20.9±0.1, 21.9±0.1, and 26.2±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least eight diffraction angles, when measured using Cu K_{α} radiation, selected from the group consisting of about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least nine diffraction angles, when measured using Cu K_{α} radiation, selected from the group consisting of about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least ten diffraction angles, when measured using Cu K_{α} radiation, selected from the group consisting of about 5.2±0.1, 8.6=0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ.

In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising diffraction angles, when measured using Cu K_{α} radiation, of about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ.

In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising diffraction angles, when measured using Cu K_{α} radiation, of about 5.2±0.1, 8.6±0.1, 10.5±0.1, 13.5±0.1, and 21.9±0.1 degrees 2θ.

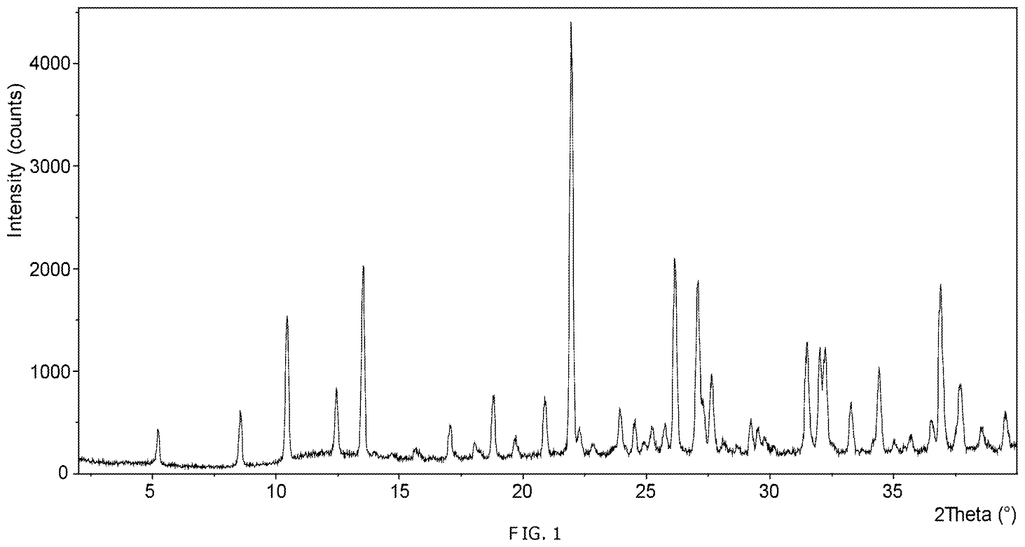

In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern substantially in accordance with FIG. 1.

In other aspects of the invention, a crystalline form of 3-chloro-7-(2-fluoro-4-(trifluoromethyl)phenyl)-2-methylbenzo[4,5] thieno[2,3-b]pyridin-4(1H)-one is provided characterised by an X-ray powder diffraction (XRPD) pattern comprising at least three diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least four diffraction angles, when measured using Cu K_{α} radiation, selected from the group consisting of about 5.9=0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least five diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least six diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least seven diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least eight diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least nine diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least ten diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least eleven diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least twelve diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ. In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising at least thirteen diffraction angles, when measured using Cu K_{α} radiation, selected from the group consisting of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ.

In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising diffraction angles, when measured using Cu K_{α} radiation, of about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ.

In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern comprising diffraction angles, when measured using Cu K_{α} radiation, of about 5.9±0.1, 9.9±0.1, 11.8±0.1, 13.9±0.1, and 14.2±0.1 degrees 2θ.

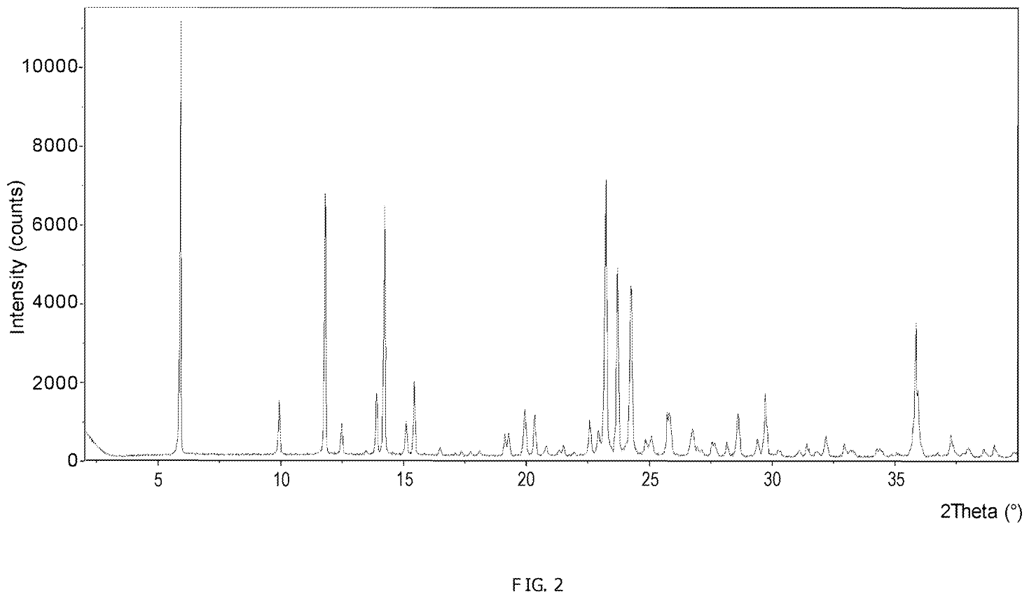

In some embodiments, the crystalline form of 3-chloro-7-(2-fluoro-4-(trifluoromethyl)phenyl)-2-methylbenzo[4,5] thieno[2,3-b]pyridin-4(1H)-one is characterised by an X-ray powder diffraction (XRPD) pattern substantially in accordance with FIG. 2.

In other embodiments, the crystalline form of 3-chloro-7-(2-fluoro-4-(trifluoromethyl)phenyl)-2-methylbenzo[4,5] thieno[2,3-b]pyridin-4(1H)-one may be a blend of crystalline forms characterised by an X-ray powder diffraction (XRPD) pattern substantially in accordance with FIGS. 1 and 2 overlapped.

### Definitions

As used herein, the term “alkyl” represents a saturated, straight, or branched hydrocarbon group. The term “C₁-C₅ alkyl” refers to an alkyl group containing from 1 to 4 carbon atoms. Example alkyls include, but are not limited to, methyl, ethyl, n-propyl, isopropyl, n-butyl, isobutyl, s-butyl, t-butyl, n-pentyl, isopentyl, neopentyl, tert-pentyl, sec-pentyl, 3-pentyl, and sec-isopentyl.

The term “hydroxyalkyl” represents an alkyl group as described above substituted with at least one hydroxy group.

The term “haloalkyl” represents an alkyl group as described above substituted with at least one halogen.

The term “halogen” represents a chloro, iodo, bromo, or fluoro group.

The term “a compound of the invention” means any one of the compounds of the invention as defined above. Specifically, the term as used herein includes but is not limited to a compound of Formula (I) or a pharmaceutically acceptable salt or tautomer thereof and is a reference to any one of the Formulas described herein, including Formula (Ia).

It will further be understood that the compounds of the invention, such as a compound of Formula (I) may exist in different tautomeric forms. Tautomers refer to isomeric forms of a compound that are in equilibrium with each other. The concentration of the isomeric forms will depend on the environment that the compound is in. For example, the compounds of the present may exhibit the following isomeric forms which are referred to as tautomers of each other:

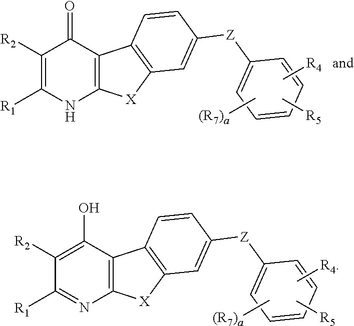

All possible tautomers are contemplated to be within the scope of the present invention. In some embodiments, any combination of tautomers may be individually or simultaneously present in a composition at any given time. Thus, in one aspect of the invention, there is provided a compound according to Formula (II):

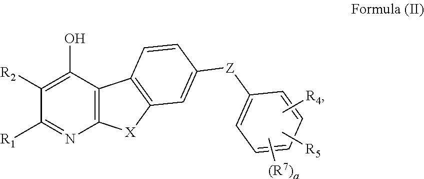

or a pharmaceutically acceptable salt thereof, with each of the substituents defined in accordance with those defined for Formula (I).

In another aspect of the invention, there is provided a compound according to (IIa):

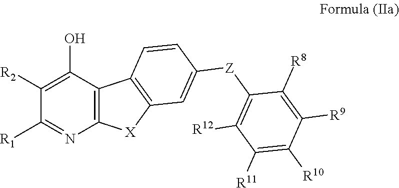

or a pharmaceutically acceptable salt thereof, with the substituents defined in accordance with Formula (Ia).

In some embodiments, the compound of the invention is selected from:
3-chloro-7-(2-fluoro-4-(trifluoromethyl)phenyl)-2-methylbenzo[4,5]thieno[2,3-b]pyridin-4-ol

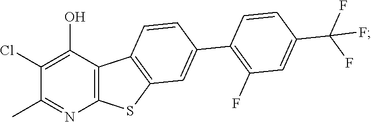 [SMILES: Cc1nc2sc3cc(-c4ccc(C(F)(F)F)cc4F)ccc3c2c(O)c1Cl]

3-chloro-7-(2-fluoro-5-(trifluoromethyl)phenyl)-2-methylbenzo[4,5]thieno[2,3-b]pyridin-4-ol

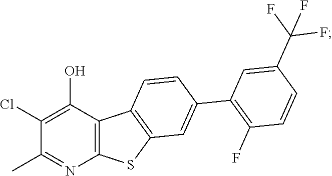 [SMILES: Cc1nc2sc3cc(-c4cc(C(F)(F)F)ccc4F)ccc3c2c(O)c1Cl]

3-chloro-2-methyl-7-(2-(trifluoromethoxy)phenyl)benzo[4,5]thieno[2,3-b]pyridin-4-ol

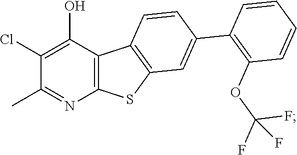 [SMILES: Cc1nc2sc3cc(-c4ccccc4OC(F)(F)F)ccc3c2c(O)c1Cl]

7-(2,4-bis(trifluoromethyl)phenyl)-3-chloro-2-methylbenzo[4,5]thieno[2,3-b]pyridin-4-ol

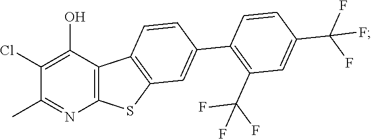 [SMILES: Cc1nc2sc3cc(-c4ccc(C(F)(F)F)cc4C(F)(F)F)ccc3c2c(O)c1Cl]

3-chloro-7-(4-(hydroxymethyl)phenyl)-2-methylbenzo[4,5]thieno[2,3-b]pyridin-4-ol

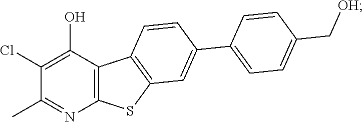 [SMILES: Cc1nc2sc3cc(-c4ccc(CO)cc4)ccc3c2c(O)c1Cl]

or
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4-ol

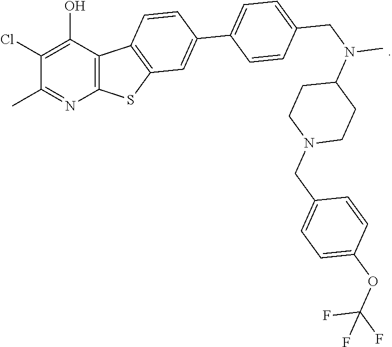 [SMILES: Cc1nc2sc3cc(-c4ccc(CN(C)C5CCN(Cc6ccc(OC(F)(F)F)cc6)CC5)cc4)ccc3c2c(O)c1Cl]

In some embodiments, the compound or composition may be a blend or equilibrium of tautomers comprising

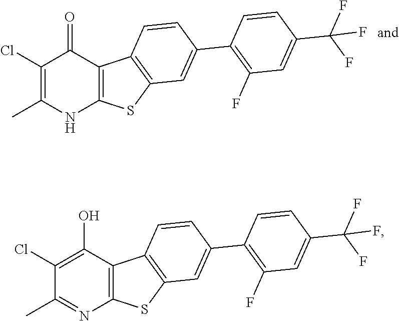 [SMILES: Cc1[nH]c2sc3cc(-c4ccc(C(F)(F)F)cc4F)ccc3c2c(=O)c1Cl ; Cc1nc2sc3cc(-c4ccc(C(F)(F)F)cc4F)ccc3c2c(O)c1Cl]

or pharmaceutically acceptable salts thereof.

It will be further understood that reference to a compound of Formula (I) or a pharmaceutically acceptable salt or tautomer thereof includes a compound of Formula (I) as a free base or as a pharmaceutically acceptable salt or tautomer thereof. Therefore, in some embodiments, the invention is directed towards a compound of Formula (I) as a free base. In other embodiments, the invention is directed to a pharmaceutically acceptable salt of a compound of Formula (I).

In some embodiments, the pharmaceutically acceptable salt of Formula (I) is a salt of Formula (III):

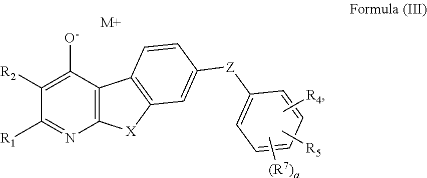

where M is a pharmaceutically acceptable counter-ion, such as a group 1 alkali metal (e.g., lithium, sodium, potassium etc), ammonium, or HTris (1,3-dihydroxy-2-(hydroxymethyl)propan-2-ammonium). In some embodiments, M is sodium.

In some embodiments, the pharmaceutically acceptable salt of Formula (I) is a salt of Formula (IIIa):

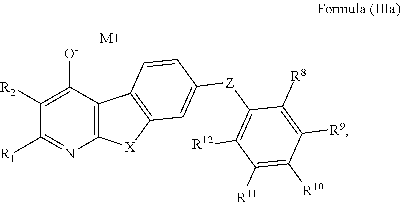

where M is as defined above.

In some embodiments, the pharmaceutically acceptable salt of Formula (I) is selected from:
sodium 3-chloro-7-[2-fluoro-4-(trifluoromethyl)phenyl]-2-methyl[1]benzothieno[2,3-b]pyridin-4-olate

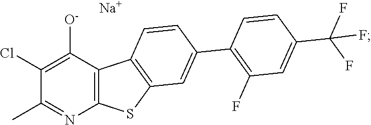 [SMILES: Cc1nc2sc3cc(-c4ccc(C(F)(F)F)cc4F)ccc3c2c([O-])c1Cl ; [Na+]]

sodium 3-chloro-7-(2-fluoro-5-(trifluoromethyl)phenyl)-2-methylbenzo[4,5]thieno[2,3-b]pyridin-4(1H)-olate

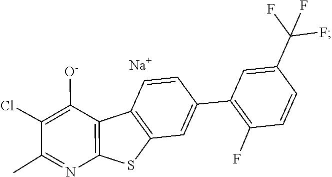 [SMILES: Cc1nc2sc3cc(-c4cc(C(F)(F)F)ccc4F)ccc3c2c([O-])c1Cl ; [Na+]]

sodium 3-chloro-2-methyl-7-(2-(trifluoromethoxy)phenyl)benzo[4,5]thieno[2,3-b]pyridin-4(1H)-olate

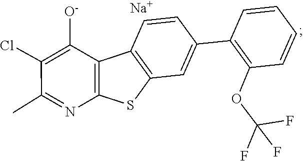 [SMILES: Cc1nc2sc3cc(-c4ccccc4OC(F)(F)F)ccc3c2c([O-])c1Cl ; [Na+]]

sodium 7-(2,4-bis(trifluoromethyl)phenyl)-3-chloro-2-methylbenzo[4,5]thieno[2,3-b]pyridin-4(1H)-olate

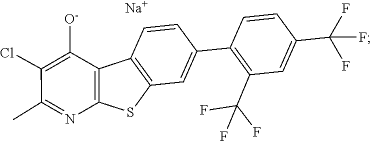 [SMILES: [Na+] ; Cc1nc2sc3cc(-c4ccc(C(F)(F)F)cc4C(F)(F)F)ccc3c2c([O-])c1Cl]

sodium 3-chloro-7-(4-(hydroxymethyl)phenyl)-2-methylbenzo[4,5]thieno[2,3-b]pyridin-4(1H)-olate

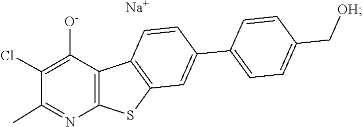 [SMILES: [Na+] ; Cc1nc2sc3cc(-c4ccc(CO)cc4)ccc3c2c([O-])c1Cl]

or
sodium 3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-olate

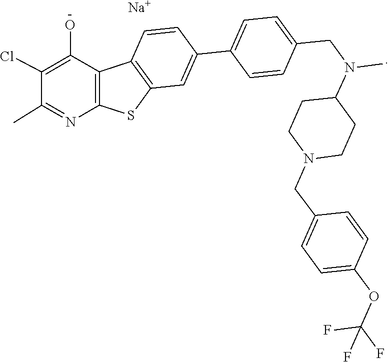

The term “pharmaceutically acceptable” refers to those compounds (including salts), materials, compositions, and dosage forms which are suitable for use contact with the tissues of humans or animals without excessive toxicity, irritation, or other side effect/complication.

Pharmaceutically acceptable salts include but are not limited to those described in Berge, J. Pharm. Sci., 1977, 66, 1-19, or those listed in PH Stahl and C G Wermuth, Handbook of Pharmaceutical Salts: Properties, Selection and Use, Second Edition, John Wiley & Sons, March 2011.

Where the compound functionality allows, suitable pharmaceutically acceptable salts of a compound of Formula (I) can be formed, which include acid or base addition salts. Acid addition salts may be formed by reaction with the appropriate acid, optionally in a suitable solvent such as an organic solvent, to give the salt which can be isolated by crystallisation and filtration. Base addition salts may be formed by reaction with the appropriate base, optionally in a suitable solvent such as an organic solvent, to give the salt which can be isolated by crystallisation and filtration.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (W-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, f-butylamine, and zinc.

In some embodiments, the compound of Formula (I) is a sodium salt or a trifluoroacetic acid salt of a compound of Formula (I).

The invention includes within its scope all possible stoichiometric and non-stoichiometric forms of the salts of compounds of Formula (I). As used herein, the term “therapeutically effective amount” means any amount which, as compared to a corresponding human subject who has not received such amount, results in improved treatment, healing, prevention, or amelioration of a disease, disorder, or side effect, or a decrease in the rate of advancement of a disease or disorder.

An appropriate “therapeutically effective amount” will depend upon a number of factors including, for example, the age and weight of the human subject, the precise condition requiring treatment and its severity, the nature of the formulation, and the route of administration, and will ultimately be at the discretion of the attendant physician.

The compounds according to Formula (I) may contain one or more asymmetric centres (also referred to as a chiral centres) and may, therefore, exist as individual enantiomers, diastereomers, or other stereoisomeric forms, or as mixtures thereof. Chiral centres, such as chiral carbon atoms, may also be present in a substituent such as an alkyl group. Where the stereochemistry of a chiral centre present in Formula (I), or in any chemical structure illustrated herein, is not specified, the structure is intended to encompass any stereoisomer and all mixtures thereof. Thus, compounds according to Formula (I) containing one or more chiral centres may be used as racemic modifications including racemic mixtures and racemates, enantiomerically-enriched mixtures, or as enantiomerically-pure individual stereoisomers.

For solvates of the compounds of the invention, or salts thereof, that are in crystalline form, the skilled artisan will appreciate that pharmaceutically acceptable solvates may be formed wherein solvent molecules are incorporated into the crystalline lattice during crystallization. Solvates may involve non-aqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The invention also includes various deuterated forms of the compounds of Formulae (I) and (Ia) or any other corresponding Formulae as defined herein, respectively, or a pharmaceutically acceptable salt or tautomer thereof. Each available hydrogen atom attached to a carbon atom may be independently replaced with a deuterium atom. A person of ordinary skill in the art will know how to synthesize deuterated forms of the compounds of Formulae (I), (Ia), respectively, or a pharmaceutically acceptable salt or tautomer thereof of the present invention. For example, deuterated materials, such as alkyl groups may be prepared by conventional techniques (see for example: methyl-c/3-amine available from Aldrich Chemical Co., Milwaukee, WI, Cat. No. 489, 689-2).

The present invention also includes isotopically-labelled compounds which are identical to those recited in Formulae (I) or (Ia), or any other corresponding Formulae as defined herein, respectively, or a pharmaceutically acceptable salt thereof but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention or pharmaceutically acceptable salts or tautomers thereof that contain the aforementioned isotopes and/or other isotopes of other atoms are within the scope of the present invention. Isotopically labelled compounds of the present invention, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Because the compounds of the present invention are intended for use in pharmaceutical compositions it will readily be understood that they are each provided in substantially pure form, for example at least 60% pure, in some aspects at least 75% pure, in some aspects at least 85% pure, and in other aspects at least 90% or 95% pure, especially at least 98% pure (% are on a weight for weight basis). Impure preparations of the compounds may be used for preparing the more pure forms used in the pharmaceutical compositions.

As used herein, the term “optionally” means that the subsequently described event(s) may or may not occur, and include both event(s) that occur and event(s) that do not occur. For example, when used in connection with the term “substituted”, i.e. “optionally substituted”, it means that the subsequently described substituents may be present or not present.

### Methods of Use

In some aspects, the invention relates to a compound of Formula (I), or a pharmaceutically acceptable salt or tautomer thereof, for use in therapy. Without wishing to be bound by theory, the compounds of the present invention are selective and specific inhibitors of the mitochondrial bc₁ complex.

Compounds of Formula (I) or a pharmaceutically acceptable salt or tautomer thereof can be useful in the treatment and/or prophylaxis of certain parasitic infections such as parasitic protozoal infections by the malarial parasite Plasmodium falciparum, species of Eimeria, Pneumocytis carinii, Trypanosoma cruzi, Trypanosoma brucei or Leishmania donovani.

In particular, compounds of Formula (I) or pharmaceutically acceptable salts or tautomers thereof can be useful in the treatment and/or prophylaxis of infection by Plasmodium falciparum. Accordingly, the invention is directed to methods of treatment and/or prophylaxis of such infections. Alternatively, the compounds of Formula (I) or pharmaceutically acceptable salts or tautomers thereof can be useful for the treatment and/or prophylaxis of infection by Plasmodium species other than Plasmodium falciparum causing human malaria. For example, the compounds of Formula (I) or pharmaceutically acceptable salts or tautomers thereof can be useful for the treatment of infection by Plasmodium Vivax, i.e., malaria caused by infection by Plasmodium Vivax.

In some embodiments, the invention relates to a compound of Formula (I) or a pharmaceutically acceptable salt or tautomer thereof for use in the treatment and/or prophylaxis of a protozoal infection. In other embodiments, the invention relates to 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof for use in the treatment and/or prophylaxis of a protozoal infection. In some embodiments, said protozoal infection is malaria or infection by Plasmodium falciparum. In some embodiments, the invention relates to a compound of Formula (I) or a pharmaceutically acceptable salt or tautomer thereof for use in the treatment and/or prophylaxis of malaria resulting from infection by Plasmodium falciparum. In some embodiments, the invention relates to 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof for use in the treatment and/or prophylaxis of malaria resulting from infection by Plasmodium falciparum.

In other aspects of the invention, there is provided a method for the treatment and/or prophylaxis of a parasitic protozoal infection, comprising administering a pharmaceutically effective amount of a compound of Formula (I) or a pharmaceutically acceptable salt or tautomer thereof to a human in need thereof. In still aspects of the invention, there is provided a method for the treatment and/or prophylaxis of a parasitic protozoal infection, comprising administering a pharmaceutically effective amount of 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof to a human in need thereof. In some embodiments, said protozoal infection is malaria or infection by Plasmodium falciparum.

In still other aspects of the invention, there is provided the use of a compound of Formula (I) or a pharmaceutically acceptable salt or tautomer thereof in the manufacture of a medicament for the treatment and/or prophylaxis of a protozoal infection. In yet other aspects of the invention, there is provided the use of a compound of 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof in the manufacture of a medicament for the treatment and/or prophylaxis of a protozoal infection. In some embodiments, said protozoal infection is malaria or infection by Plasmodium falciparum. Accordingly, a compound of Formula (I) or a pharmaceutically acceptable salt or tautomer thereof may be used in the treatment and/or prophylaxis of malaria. Therefore, the invention also relates to a method for the treatment and/or prophylaxis of malaria comprising administering a pharmaceutically effective amount of a compound of Formula (I) or a pharmaceutically acceptable salt or tautomer thereof to a human in need thereof. In addition, the invention relates to the use of a compound of Formula (I) or a pharmaceutically acceptable salt or tautomer thereof in the manufacture of a medicament for the treatment and/or prophylaxis of malaria. Also, the invention relates to the use of a compound of 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof in the manufacture of a medicament for the treatment and/or prophylaxis of malaria.

It will be appreciated by those skilled in the art that references herein to treatment refer to the treatment of established conditions, such as malaria. However, compounds of the invention may also be useful in the prevention of such diseases, such as in the prevention of malaria. Thus, in some embodiments, there is provided the treatment or prevention of a disease such as malaria. In another embodiment, there is provided the treatment of a disease such as malaria. In a further embodiment, there is provided the prevention of a disease such as malaria.

In some embodiments, the malaria is multi-drug resistant malaria. Therefore, in some embodiments, a compound of Formula (I) or a pharmaceutically acceptable salt or tautomer thereof may be useful in the treatment and/or prophylaxis of sensitive and/or multi-drug resistant malaria. In other embodiments, 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof may be useful in the treatment and/or prophylaxis of sensitive and/or multi-drug resistant malaria.

### Pharmaceutical Compositions

The compounds of Formula (I) and pharmaceutically acceptable salts and tautomers thereof will normally, but not necessarily, be formulated into pharmaceutical compositions prior to administration to a patient. Accordingly, in another aspect there is provided a pharmaceutical formulation comprising (a) a compound of Formula (I), or a pharmaceutically acceptable salt or tautomer thereof; and (b) a pharmaceutically acceptable excipient or carrier.

Suitable pharmaceutically acceptable excipients include the following types of excipients: binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation. The carrier excipient must be “acceptable” in the sense of being compatible with the other ingredients of the formulation and not deleterious to the recipient thereof. Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In some aspects, the invention is directed to a solid or liquid oral dosage form such as a liquid, tablet, lozenge or a capsule, comprising a safe and effective amount of a compound of the invention and a carrier. The carrier may be in the form of a diluent or filler. Suitable diluents and fillers in general include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. A liquid dosage form will generally consist of a suspension or solution of the compound or pharmaceutically acceptable salt or tautomer in a liquid carrier for example, ethanol, olive oil, glycerine, glucose (syrup) or water (e.g. with an added flavouring, suspending, or colouring agent). Where the composition is in the form of a tablet or lozenge, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers or a semi solid e.g. mono di-glycerides of capric acid, Gelucire and Labrasol, or a hard-capsule shell e.g. gelatin. Where the composition is in the form of a soft-shell capsule e.g. gelatin, any pharmaceutical carrier routinely used for preparing dispersions or suspensions may be considered, for example aqueous gums or oils, and may be incorporated in a soft capsule shell.

Pharmaceutical compositions may be administered by any appropriate route, for example by the oral (including buccal or sublingual), inhaled, intranasal, topical (including buccal, sublingual or transdermal), parenteral (including subcutaneous, intramuscular, intravenous or intradermal) route. In particular, pharmaceutical compositions are administered via an oral route of administration. Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

An oral solid dosage form may further comprise an excipient in the form of a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise an excipient in the form of a disintegrant. Suitable disintegrants include crospovidone, sodium starch glycolate, croscarmellose, alginic acid, and sodium carboxymethyl cellulose. The oral solid dosage form may further comprise an excipient in the form of a lubricant. Suitable lubricants include stearic acid, magnesium stearate, calcium stearate, and talc.

In some embodiments, the compound of Formula (I) or pharmaceutically acceptable salt or tautomer thereof is prepared for administration by injection, either intramuscularly or subcutaneously. In one aspect, the present invention relates to an injectable composition comprising the compound of Formula (I) or pharmaceutically acceptable salt or tautomer thereof. Standard formulation and manufacturing techniques can be used to produce a suitable stable, sterile vehicle for injection containing the compound of Formula (I) or pharmaceutically acceptable salt or tautomer thereof of the present invention. The injectable pharmaceutical composition is a long-acting injectable composition and provides a controlled release of the compound of Formula (I) or pharmaceutically acceptable salt or tautomer thereof.

In some embodiments, the compound of Formula (I) or pharmaceutically acceptable salt or tautomer thereof is prepared for administration by injection, with the composition comprising the compound or pharmaceutically acceptable salt and a pharmaceutically acceptable excipient or carrier, such as Tween 20, PEG400 and/or mannitol. In some embodiments, the compound of Formula (I) or pharmaceutically acceptable salt or tautomer thereof is formulated with Tween 20, PEG400 and mannitol, and suitable as a long-acting injectable composition. The composition may or may not be buffered.

In some embodiments, the compound of Formula (I) or pharmaceutically acceptable salt or tautomer thereof is prepared for administration by injection, with the composition comprising compound or pharmaceutically acceptable salt and a pharmaceutically acceptable excipient or carrier, such as poloxamer P338 and PEG300. The composition may or may not be buffered.

In some embodiments, the invention provides a pharmaceutical composition comprising an aqueous, injectable suspension comprising a compound of Formula (I) or pharmaceutically acceptable salt or tautomer thereof, in crystalline form and at a concentration of 10-1000 mg/ml, 10-500 mg/mL, 100-500 mg/mL, 100-400 mg/mL, 200-700 mg/ml, 200-500 mg/mL, 200-400 mg/ml, 300-900 mg/mL, 300-700 mg/mL, 300-500 mg/mL, or 200-350 mg/mL, and in some embodiments at a concentration of approximately 100 mg/mL, 150 mg/mL, 200 mg/mL, 250 mg/mL, 300 mg/mL, 350 mg/mL, 400 mg/mL, 450 mg/mL, or 500 mg/mL. The crystals may have (a) a micron particle size distribution (D10: about 1 μm, D50: about 3 μm, D90: about 5 μm) or (b) a sub-micron particle size distribution (D10: about 0.1 μm, D50: about 0.2 μm, D90: about 0.5 μm). As used herein, “D10: about 1 μm” is defined to mean ten percent of the particles are about 1 μm or smaller, “D50: about 3 μm” is defined to mean fifty percent of the particles are about 3 μm or smaller, etc. In some cases, the pharmaceutical composition comprises pharmaceutically acceptable excipients, such as one or more of the following:
Tween 20 (1.5-4.5% w/w) or Poloxamer 338 (3-4.5% w/w) may be used as wetting agent to disperse and stabilize micron or sub-micron drug particles in aqueous vehicle.
PEG 3350 (1.5-4.5% w/w) or sodium carboxymethyl cellulose (0.33-0.99% w/w) may be used as stabilizing agent to stabilize micron or sub-micron drug particles in the aqueous vehicle.
Mannitol (2-3.5% w/w) may be used as tonicity adjuster to make the suspension isotonic with physiological fluid.
A buffering agent may be used in the formulation at quantity sufficient levels to maintain, either target pH of 6.5-7.5 (via phosphate buffer inclusion) or target pH of 4-5.5 (via acetate buffer inclusion).

In some cases, the injectable suspension is terminally sterilized using either moist heat sterilization (autoclave) or ionizing radiation (gamma irradiation) methods. An aqueous injectable suspension comprising a micron particle size distribution, as mentioned above, may be manufactured via either a homogenization technique (using micronized API as the input) or a wet bead milling technique (input API milled to the micron specification mentioned above). In some embodiments, the particle size of the crystalline Formula (I) compound or 3-chloro-7-(2-fluoro-4-(trifluoromethyl)phenyl)-2-methylbenzo[4,5] thieno[2,3-b]pyridin-4(1H)-one may be from about 1 μm to about 100 μm, from about 1 μm to about 50 μm, from about 1 μm to about 25 μm, from about 1 μm to about 10 μm, from about 1 μm to about 9 μm, from about 1 μm to about 8 μm, from about 1 μm to about 7 μm, from about 1 μm to about 6 μm, from about 1 μm to about 5 μm, from about 1 μm to about 4 μm, from about 1 μm to about 3 μm, from about 1 μm to about 2 μm, from about 2 μm to about 8 μm, from about 2 μm to about 6 μm, from about 2 μm to about 4 μm, from about 3 μm to about 9 μm, from about 3 μm to about 7 μm, from about 3 μm to about 5 μm, from about 4 μm to about 10 μm, from about 4 μm to about 8 μm, from about 4 μm to about 6 μm, from about 4 μm to about 5 μm, from about 5 μm to about 10 μm, from about 5 μm to about 8 μm, from about 5 μm to about 6 μm, or any combinations thereof.

An aqueous injectable suspension comprising a sub-micron particle size distribution, as mentioned above, may be manufactured via a wet bead milling technique (input API milled to sub-micron specification mentioned above). In some embodiments, the particle size of the crystalline Formula (I) compound or 3-chloro-7-(2-fluoro-4-(trifluoromethyl)phenyl)-2-methylbenzo[4,5] thieno[2,3-b]pyridin-4(1H)-one may be from about 0.1 μm to about 0.001 μm, from about 0.1 μm to about 0.01 μm, from about 0.1 μm to about 1 μm, from about 0.1 μm to about 0.9 μm, from about 0.1 μm to about 0.8 μm, from about 0.1 μm to about 0.7 μm, from about 0.1 μm to about 0.6 μm, from about 0.1 μm to about 0.5 μm, from about 0.1 μm to about 0.4 μm, from about 0.1 μm to about 0.3 μm, from about 0.1 μm to about 0.2 μm, from about 0.2 μm to about 0.8 μm, from about 0.2 μm to about 0.6 μm, from about 0.2 μm to about 0.4 μm, from about 0.3 μm to about 0.9 μm, from about 0.3 μm to about 0.7 μm, from about 0.3 μm to about 0.5 μm, from about 0.4 μm to about 1 μm, from about 0.4 μm to about 0.8 μm, from about 0.4 μm to about 0.6 μm, from about 0.4 μm to about 0.5 μm, from about 0.5 μm to about 1 μm, from about 0.5 μm to about 0.8 μm, from about 0.5 μm to about 0.6 μm, or any combinations thereof.

When a compound of Formula (I) or pharmaceutically acceptable salt or tautomer thereof is used in the treatment and/or prophylaxis of malaria, or Plasmodium falciparum, it may be employed alone or in combination with at least one other therapeutic agent, such as at least one other anti-parasitic agents, for example an anti-malarial agent.

In an embodiment, there is provided a combination of (a) a compound of Formula (I) or a pharmaceutically acceptable salt or tautomer thereof; and (b) at least one other therapeutic agent.

In some embodiments, the at least one other therapeutic agent is an anti-fungal agent, such as ketoconazole, itraconazole, fluconazole, fosfluconazole, voriconazole, posaconazole, and isavuconazole, griseofulvin or terbinafine.

In some embodiments, the present invention relates to a combination of (a) a compound of Formula (I) or a pharmaceutically acceptable salt or tautomer thereof; and (b) at least one other anti-malarial agent. In other embodiments, the present invention relates to a combination of (a) 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof; and (b) at least one other anti-malarial agent.

In an embodiment, the combination comprises one or two or three additional anti-malarial agents. For the avoidance of doubt, the at least one other anti-malarial agent is not a compound of Formula (I).

The at least one other anti-malarial agent is an agent in development, approved of recommended for the treatment and/or prophylaxis of malaria.

The at least one other anti-malarial agent may be selected from the group consisting of chloroquine, mefloquine, primaquine, pyrimethamine, quinine, artemisinin, halofantrine, doxycycline, amodiaquine, atovaquone, tafenoquine, dapsone, proguanil, sulfadoxine, cycloguanil, fansidar, piperaquine, lumefantrine, artesunate, dihydroartemisinin, arthemeter, fosmidomycin and azithromycin.

The at least one other anti-malarial agent may be tafenoquine.

In an embodiment, the additional anti-malarial agents are atovaquone and proguanil. The at least one other anti-malarial agent may also be selected from the group consisting of ferroquine, KAF156, cipargamin, DSM265, artemisone, artemisinin, artefenomel, MMV048, SJ733, P218, MMV253, PA92, DDD498, AN13762, DSM421, UCT947, ACT 451840, 6-chloro-7-methoxy-2-methyl-3-{4-[4-(trifluoromethoxy)phenoxy]phenyl}quinolin-4(1H)-one, 6-chloro-7-methoxy-2-methyl-3-(4-(4-(trifluoromethoxy)phenoxy)phenyl)quinolin-4(1H)-one, a pharmaceutically salt thereof, and a combination thereof. In some embodiments, the additional anti-malarial agent is 6-chloro-7-methoxy-2-methyl-3-{4-[4-(trifluoromethoxy)phenoxy]phenyl}quinolin-4(1H)-one, 6-chloro-7-methoxy-2-methyl-3-(4-(4-(trifluoromethoxy)phenoxy)phenyl)quinolin-4(1H)-one, a pharmaceutically salt thereof, or a combination thereof.

The at least one other anti-malarial agent may also be selected from the group consisting of OZ609, OZ277 and SAR97276.

In the treatment and/or prophylaxis of Plasmodium falciparum infections, the at least one or two or three additional anti-malarial agents are selected as follows, wherein at least one of the anti-malarial agents is an artemisinin-based agent:
artemether+lumefantrine
artesunate+amodiaquine
artesunate+mefloquine
dihydroartemisinin+piperaquine
artesunate+sulfadoxine-pyrimethamine (SP)

The above combination treatments are known as artemisinin-based combination therapies (ACTs). The choice of ACT is usually based on the results of therapeutic efficacy studies against local strains of Plasmodium falciparum malaria.

In the treatment and/or prophylaxis of Plasmodium vivax infections, an ACT may be used, as described above. Alternatively, the at least one other anti-malarial agent may be chloroquine, particularly in areas without chloroquine resistant Plasmodium vivax. In areas where resistant Plasmodium vivax has been identified, infections may be treated with an ACT, as described above.

The combinations may conveniently be presented for use in the form of a pharmaceutical composition or formulation. Therefore, also contemplated herein is a pharmaceutical composition comprising (a) a compound of Formula (I) or a pharmaceutically acceptable salt or tautomer thereof, as herein described, together with (b) at least one other anti-malaria agent and (c) one or more pharmaceutically acceptable excipients as herein described.

A compound of Formula (I) or pharmaceutically acceptable salt or tautomer thereof and at least one other therapeutic agent may be administered together or separately and, when administered separately, this may occur separately or sequentially in any order (by the same or by different routes of administration).

### Long-Acting Injectable Formulation

In some aspects of the present invention, the compound of Formula (I) or a pharmaceutically acceptable salt or tautomer thereof is formulated as a long-acting injectable formulation. In other aspects of the present invention, 3-chloro-7-(2-fluoro-4-(trifluoromethyl)phenyl)-2-methylbenzo[4,5] thieno[2,3-b]pyridin-4(1H)-one or a pharmaceutically acceptable salt or tautomer thereof is formulated as a long-acting injectable formulation. These may be administered intravenously (IV), intramuscularly (IM), or subcutaneously (SC).

In some embodiments, the long-acting injectable formulation includes 100 mg-1000 mg of a compound of Formula (I) or 3-chloro-7-(2-fluoro-4-(trifluoromethyl)phenyl)-2-methylbenzo[4,5] thieno[2,3-b]pyridin-4(1H)-one, and provides prophylactic treatment against malaria for up to one month, for up to two months, for up to three months, for up to four months, for up to five months, or for up to six months. In other embodiments, the long-acting injectable formulation includes 100 mg-1000 mg of a compound of Formula (I) or 3-chloro-7-(2-fluoro-4-(trifluoromethyl)phenyl)-2-methylbenzo[4,5] thieno[2,3-b]pyridin-4(1H)-one, and provides prophylactic treatment against malaria for up to three months. In some embodiments, the long-acting injectable formulation includes 350 mg of a compound of Formula (I) or 3-chloro-7-(2-fluoro-4-(trifluoromethyl)phenyl)-2-methylbenzo[4,5] thieno[2,3-b]pyridin-4(1H)-one, and will provide prophylactic treatment against malaria for up to one month, for up to two months, for up to three months, or for up to four months for up to five months, or for up to six months . . . .

In some embodiments, the long-acting injectable formulation includes a compound which is:
3-chloro-7-(2-fluoro-4-(trifluoromethyl)phenyl)-2-methylbenzo[4,5] thieno[2,3-b]pyridin-4(1H)-one;
sodium 3-chloro-7-[2-fluoro-4-(trifluoromethyl)phenyl]-2-methyl[1]benzothieno[2,3-b]pyridin-4-olate;
3-chloro-7-(2-fluoro-5-(trifluoromethyl)phenyl)-2-methylbenzo[4,5]thieno[2,3-b]pyridin-4(1H)-one;
3-chloro-2-methyl-7-(2-(trifluoromethoxy)phenyl)benzo[4,5]thieno[2,3-b]pyridin-4(1H)-one; 7-(2,4-bis(trifluoromethyl)phenyl)-3-chloro-2-methylbenzo[4,5]thieno[2,3-b]pyridin-4(1H)-one;
3-chloro-7-(4-(hydroxymethyl)phenyl)-2-methylbenzo[4,5]thieno[2,3-b]pyridin-4(1H)-one;
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one; or
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one 2,2,2-trifluoroacetate,
   and pharmaceutically acceptable salts thereof.

In some embodiments, the long-acting injectable formulation includes 100 mg-1000 mg (in some aspects 350 mg) of a compound which is:
3-chloro-7-(2-fluoro-4-(trifluoromethyl)phenyl)-2-methylbenzo[4,5] thieno[2,3-b]pyridin-4(1H)-one;
sodium 3-chloro-7-[2-fluoro-4-(trifluoromethyl)phenyl]-2-methyl[1]benzothieno[2,3-b]pyridin-4-olate;
3-chloro-7-(2-fluoro-5-(trifluoromethyl)phenyl)-2-methylbenzo[4,5]thieno[2,3-b]pyridin-4(1H)-one;
3-chloro-2-methyl-7-(2-(trifluoromethoxy)phenyl)benzo[4,5]thieno[2,3-b]pyridin-4(1H)-one;
7-(2,4-bis(trifluoromethyl)phenyl)-3-chloro-2-methylbenzo[4,5]thieno[2,3-b]pyridin-4(1H)-one;
3-chloro-7-(4-(hydroxymethyl)phenyl)-2-methylbenzo[4,5]thieno[2,3-b]pyridin-4(1H)-one;
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one; or
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one 2,2,2-trifluoroacetate,
and pharmaceutically acceptable salts.

In some embodiments, the long-acting injectable composition includes 350 mg of 3-chloro-7-(2-fluoro-4-(trifluoromethyl)phenyl)-2-methylbenzo[4,5] thieno[2,3-b]pyridin-4(1H)-one. In some embodiments, the long-acting injectable composition includes 350 mg of 3-chloro-7-(2-fluoro-4-(trifluoromethyl)phenyl)-2-methylbenzo[4,5] thieno[2,3-b]pyridin-4(1H)-one, and is capable of providing prophylactic treatment against malaria for up to 2 months or up to 3 months.

In some embodiments, the long-acting injectable composition includes 3-chloro-7-(2-fluoro-4-(trifluoromethyl)phenyl)-2-methylbenzo[4,5] thieno[2,3-b]pyridin-4(1H)-one capable of providing prophylactic treatment against malaria for up to 1 month, for up to 2 months, up to 3 months, up to 4 months, up to 5 months, or up to 6 months.

In some embodiments, the ability of the long-acting injectable composition to provide prophylactic treatment against malaria for up to one, two, three, four, five, or six months depends on the concentration and/or the micron or sub-micron particle size distribution of the crystalline Formula (I) or crystalline 3-chloro-7-(2-fluoro-4-(trifluoromethyl)phenyl)-2-methylbenzo[4,5] thieno[2,3-b]pyridin-4(1H)-one. By carefully designing the particle size using either a homogenization technique (using micronized API as the input) or a wet bead milling technique (input API milled to the micron specification mentioned above), the long-acting injectable composition can be engineered in combination with the formulation for a desired one, two, three, four, five, or six month prophylactic treatment against malaria time. The various exemplary particle size distributions are listed and provided above.

### Dosages

The amount of a compound of the invention or pharmaceutically acceptable salt thereof or tautomer and the further therapeutically active agent(s) and the relative timings of administration will be selected in order to achieve the desired combined therapeutic effect, and can be subject to the judgement of a health-care practitioner.

Typical amounts administered will be 100 mg-1000 mg, in some aspects 350 mg, and will provide prophylactic treatment against malaria for 2 months (in some aspects up to 3 months).

A typical daily dose of the compound of Formula (I) can be in the range from 100 μg to 100 mg per kg of body weight, more typically 5 ng to 25 mg per kg of bodyweight, and more usually 10 ng to 15 mg per kg (e.g. 10 ng to 10 mg, and more typically 1 mg per kg to 20 mg per kg, for example 1 mg to 10 mg per kg) of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days, or in some aspects once every 1, 2, or 3 months, in some aspects once in a 3 month period (i.e. 90 days) for example. In some embodiments, the compound of Formula (I) is administered once in a 90 day period. In some embodiments, 100 mg-1000 mg of a compound of Formula (I) is administered in a single administration. In some aspects, in some aspects 350 mg of a compound of Formula (I) is administered in a single administration. In some aspects, 100 mg-1000 mg of the compound of Formula (I) is administered, and will provide prophylactic treatment against malaria for 1 months (in some aspects up to 3 months). In some aspects, 100 mg-1000 mg of the compound of Formula (I) is administered, and will provide prophylactic treatment against malaria for 2 months (in some aspects up to 3 months). In some aspects, 100 mg-1000 mg of the compound of Formula (I) is administered, and will provide prophylactic treatment against malaria for 3 months. In one aspect, 350 mg of a compound of Formula (I) is administered and provides prophylactic treatment against malaria for 2 months (in some aspects up to 3 months).

Dosages may also be expressed as the amount of drug administered relative to the body surface area of the patient (mg/m²). A typical daily dose of the compound of formula (I) can be in the range from 3700 pg/m² to 3700 mg/m², more typically 185 ng/m² to 925 mg/m², and more usually 370 ng/m² to 555 mg/m² (e.g. 370 ng/m² to 370 mg/m², and more typically 37 mg/m² to 740 mg/m², for example 37 mg/m² to 370 mg/m²) although higher or lower doses may be administered where required. The compound of the formula (I) can be administered on a daily basis or on a repeat basis every 2, or 3, or 4, or 5, or 6, or 7, or 10 or 14, or 21, or 28 days, or in some aspects once every 1,2, or 3 months, in other aspects once in a 3 month period for example.

The compounds of the invention may be administered orally in a range of doses, for example 0.1 to 5000 mg, or 1 to 1500 mg, 2 to 800 mg, or 5 to 500 mg, e.g. 2 to 200 mg or 10 to 1000 mg, particular examples of doses including 10, 20, 50, 80 mg, and 350 mg.

In one particular dosing schedule, a patient will be given an injection of a compound of the formula (I) once, and the treatment will provide prophylactic treatment against malaria for a period of up to 1 month, in some aspects up to 2 months or in other aspects up to 3 months.

Ultimately, however, the quantity of compound administered, and the type of composition used will be commensurate with the nature of the disease or physiological condition being treated and will be at the discretion of the physician.

Determination of effective dosages in this context is typically based on animal model studies followed up by human clinical trials and is guided by administration protocols that significantly reduce the occurrence or severity of targeted disease symptoms or conditions in the human. Suitable models in this regard include, for example, murine, rat, avian, porcine, feline, non-human primate, and other accepted animal model subjects known in the art. Alternatively, effective dosages can be determined using in vitro models (for example, whole cell assays that monitor the effect of various drugs on parasite growth rate). Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease).

In all of the above-described uses, the administered form of the compound of Formula (I) or pharmaceutically acceptable salt or tautomer thereof the compounds have low solubility and very low intrinsic clearance, and thus provide long-lasting chemoprotection against malaria.

### General Synthetic Routes

The compounds of the invention may be made in a variety of methods, which include those methods conventionally known in the field of chemistry. Any previously defined variable will continue to have the previously defined meaning unless otherwise indicated. Illustrative general synthetic methods are set out in the following schemes, and can be readily adapted to prepare the compounds of the invention. Specific compounds prepared according to the experimental procedure are disclosed in the Examples Section.

Compounds of Formula (I) above may be prepared from brominated intermediate compounds of Formula (A):

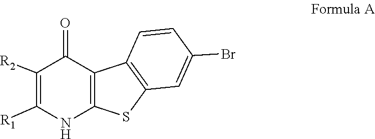

wherein R¹ and R² are as defined in Formula (I) above. Intermediate compounds of Formula (A) may be prepared by using scheme 1, which is shown for compounds containing R¹ as methyl and R² as chloro for the purposes of illustration. However, the skilled artisan would be readily capable of modifying the scheme to prepare the compounds of Formula (A).

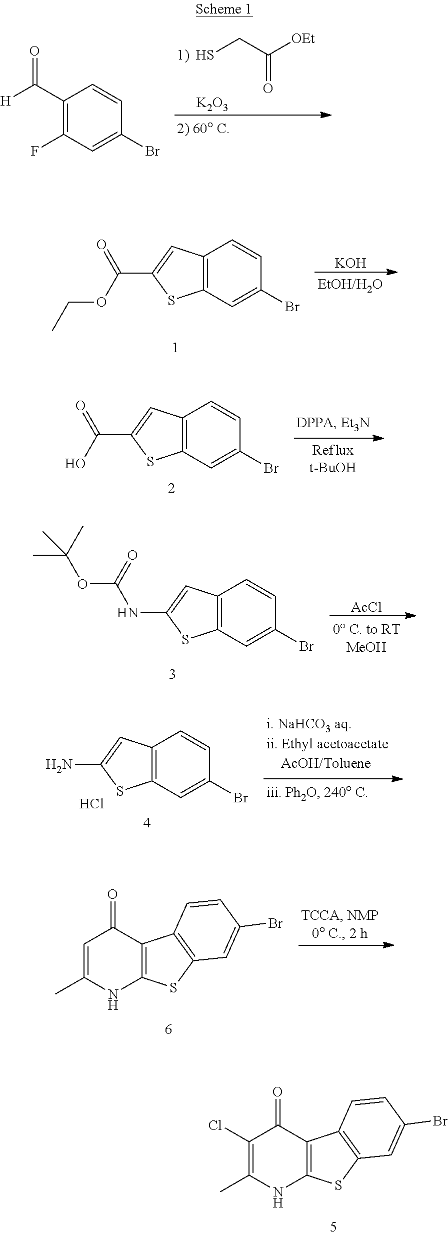

A nucleophilic displacement of the aromatic fluorine and base-induced ring-closure was performed in the presence of thioglycolate and K₂CO₃ in DMF, leading to 6-bromo-1-benzothiophene-2-carboxylate 1. Saponification of the ethyl ester 1 with potassium hydroxide in a mixture 1:1 EtOH/H₂O easily gave the benzothiophene carboxylic acid 2. Further, Curtius rearrangement with DPPA in t-BuOH provided the W-Boc aniline derivative 3. Boc cleavage using acetyl chloride in MeOH at 0° C., led to the benzothiophene amine hydrochloride 4. Previously prepared hydrochloride amine 4 was treated with NaHCO₃aq. and the free amine obtained reacted with ethyl acetoacetate to afford the corresponding enamine intermediate, which then cyclized by heating in diphenyl ether to lead the desired tricyclic scaffold 5. The benzothienopyridinone 5 was subsequently chlorinated with TCCA to lead 6.

Intermediate 8 may be by the procedure illustrated in scheme 2:

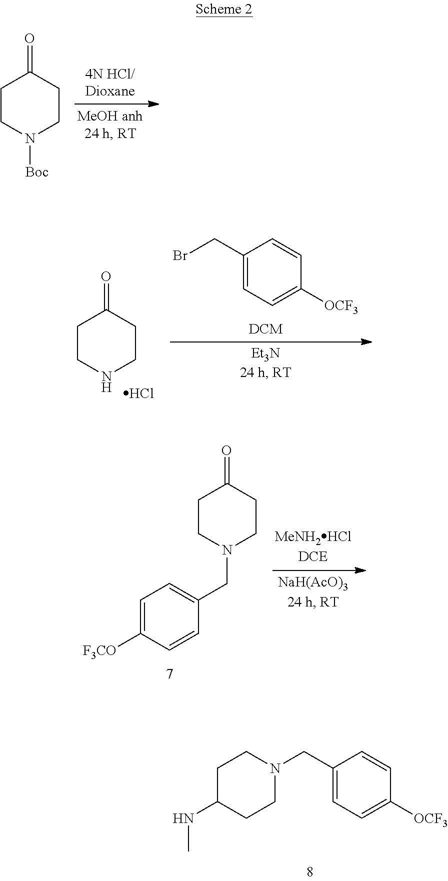 [SMILES: CC(C)(C)OC(=O)N1CCC(=O)CC1 ; O=C1CCNCC1 ; FC(F)(F)Oc1ccc(CBr)cc1 ; Cl ; O=C1CCN(Cc2ccc(OC(F)(F)F)cc2)CC1 ; CNC1CCN(Cc2ccc(OC(F)(F)F)cc2)CC1]

Intermediate 8 was prepared from the tert-butyl 4-oxopiperidine-1-carboxylate. Boc deprotection and further alkylation with 4-(trifluoromethoxy)benzyl bromide and Et₃N led to the corresponding benzyl-4-piperidone which after reductive amination with methylamine hydrochloride afforded the secondary amine 8.

The examples 1-6 may be prepared either by method A or method B illustrated in in scheme 3, with R¹ as methyl and R² as chloro for the purposes of illustration. However, the skilled artisan would be readily capable of modifying the scheme to prepare the compounds of Formula (II).

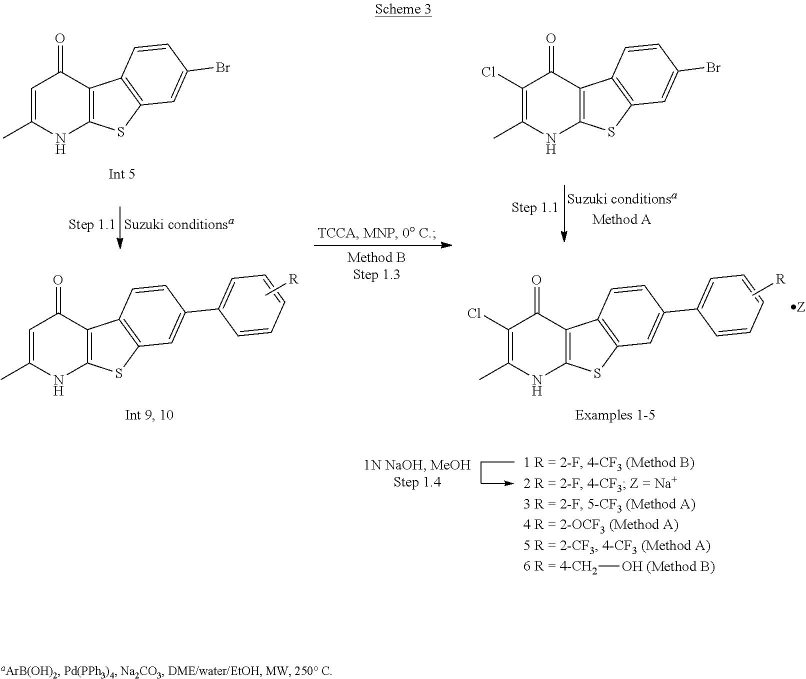

The Suzuki conditions in Step 1.1 and 1.2 of Scheme 1 may be performed by reaction in typically a mixture of DME/water/EtOH, under pressure e.g. at up to 16 bar, and at a temperature typically of 250° C. Step 1.3 may be performed in N-methyl pyrrolidone with TCCA. In step 1.4 the sodium salt may be prepared by addition of aqueous sodium hydroxide to a suspension of the product of the preceding step.

Example 7 may be prepared by the procedure illustrated in scheme 4.

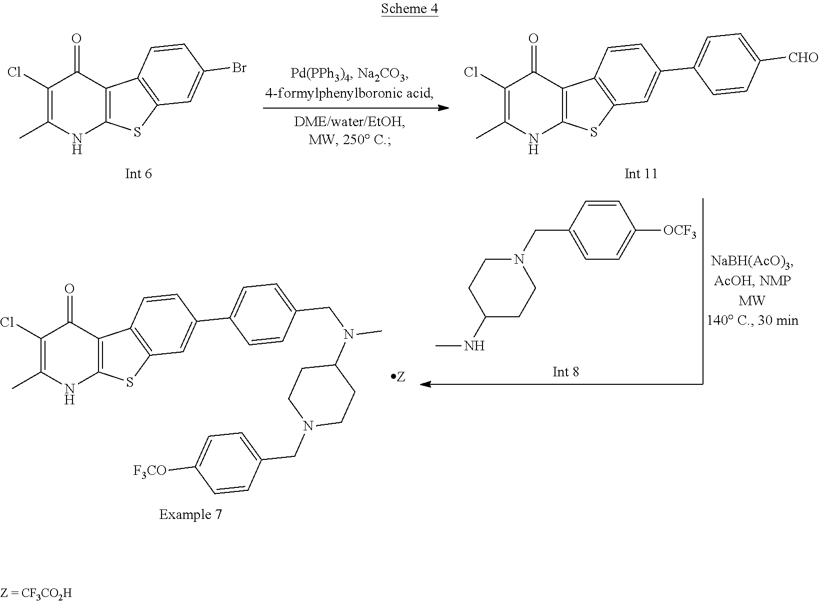

Example 7 was prepared via reductive amination of the aldehyde intermediate 11 and the corresponding secondary amines intermediate 8. Aldehyde 11 was prepared via the Suzuki coupling reaction previously described. Further reductive amination with the corresponding N-methylpiperidin-4-amine and NaBH(AcO)₃ in NMP required microwave conditions to be completed (140° C., 30 min) and Example 7 was isolated as the corresponding TFA salts after preparative HPLC purification. 10 Example 1 may also be prepared by the procedure illustrated in Scheme 5.

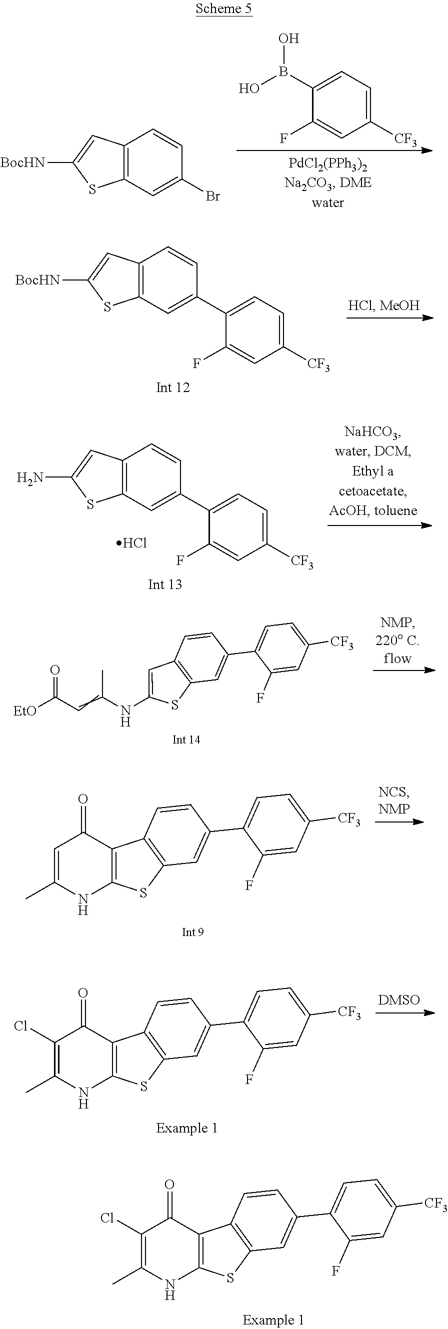

### EXAMPLES

The invention will now be illustrated by way of the following non-limiting examples. While particular embodiments of the invention are described below a skilled artisan will appreciate that various changes and modifications can be made. References to preparations carried out in a similar manner to, or by the general method of, other preparations, may encompass variations in routine parameters such as time, temperature, work-up conditions, and minor changes in reagent amounts, etc.

In certain of the following intermediates and examples, starting materials are identified by reference to other intermediate or example numbers. This does not signify that the actual material from any particular intermediate or example was necessarily used in a subsequent step exemplified herein, but is used as a short-hand means of denoting the relevant compound.

Where materials were commercially available, this is indicated in parentheses after the compound name in capitals. Commercial reagents and solvents were used as received. All solvents used in the reaction were high purity grade or anhydrous grade. Proton nuclear magnetic resonance (¹H NMR) spectra were recorded, and chemical shifts are reported in parts per million (δ) downfield from the internal standard tetramethylsilane (TMS). Abbreviations for NMR data are as follows: s=single, d=doublet, t=triplet, q=quartet, m=multiplet, dd=doublet of doublets, dt=doublet of triplets, app=apparent, br=broad. Mass spectra were obtained using electrospray (ES) ionisation techniques. All temperatures are reported in degrees Centigrade.

Where diastereomers are represented and the absolute stereochemistry is known, the stereocentre, i.e. the chiral carbon atom, is labelled with R or S.

### Compound Preparation

### Intermediate 1: Ethyl 6-bromo-1-benzothiophene-2-carboxylate

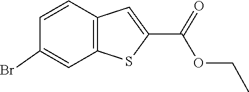

To a solution of 4-bromo-2-fluorobenzaldehyde (10 g, 9.26 mmol) in dry DMF (101.8 mL, 2 mL/mmol), K₂CO₃ (10.21 g, 1.5 eq) was added. The mixture was cooled to 0° C. under nitrogen atmosphere and ethyl thioglycolate (5.38 mL, 1 eq) was added dropwise. The reaction mixture was allowed to warm to room temperature overnight. After 20 h the mixture was heated at 65-70° C. for 6 h. The reaction was treated at room temperature with H₂O until completed precipitation of a solid, which was filtered, washed with H₂O and dried under vacuum to afford 12.65 g of the titled compound as a yellow solid. Yield 90%. ¹H NMR (300 MHZ, CDCl₃) d 8.04-7.97 (m, 1H), 7.73 (d, J=8.49 Hz, 1H), 7.51 (dd, J=1.68, 8.57 Hz, 1H), 4.41 (q, J=7.13 Hz, 2H), 1.46-1.36 (t, J=7.13 Hz, 3H). ¹³C NMR (101 MHZ, DMSO-d₆) δ 162.21, 143.26, 137.97, 134.31, 130.87, 128.97, 127.97, 126.00, 121.14, 62.10, 14.60. ESIMS m/z: 285 [M+H]⁺. Purity was determined as >95% by HPLC (393 nm), Rt: 4.07 min (Sunfire 3.5μ, C18 4.6×50 mm, ammonium acetate 0.05% pH 7/Acetonitrile).

### Intermediate 2: 6-bromo-1-benzothiophene-2-carboxylic acid

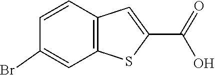 [SMILES: O=C(O)c1cc2ccc(Br)cc2s1]

Intermediate 1 (12.65 g, 49.37 mmol) was suspended in a 1:1 mixture of EtOH/H₂O (100 mL, 2 mL/mmol) and KOH (12.44 g, 5 eq.) was added. The mixture was refluxed for 1.5 h and was allowed to warm to room temperature, then was acidified with conc. HCl. The resulting solid was filtered, washed with H₂O and dried under vacuum to afford 11.22 g of the titled compound as an orange solid. Yield 98%. ¹H NMR (300 MHZ, DMSO-d₆) δ 13.57 (br. s., 1H), 8.36 (d, J=1.76 Hz, 1H), 8.10 (s, 1H), 7.94 (d, J=8.64 Hz, 1H), 7.61 (dd, J=1.83, 8.57 Hz, 1H). ¹³C NMR (101 MHZ, DMSO-d₆) δ 163.73, 143.32, 138.19, 136.11, 130.31, 128.79, 127.80, 125.94, 120.78. ESIMS m/z: 257 [M+H]⁺. Purity was determined as >95% by HPLC (231 nm), Rt: 1.98 min (Sunfire 3.5μ, C18 4.6×50 mm, ammonium acetate 0.05% pH 7/Acetonitrile).

### Intermediate 3: 1,1-dimethylethyl (6-bromo-1-benzothien-2-yl)carbamate

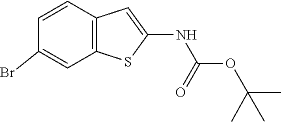 [SMILES: CC(C)(C)OC(=O)Nc1cc2ccc(Br)cc2s1]

To a solution of intermediate 2 (11.22 g, 43.64 mmol) in 2-methyl-2-propanol (220 mL, 5 mL/mmol) were added portionwise DPPA (10.52 mL, 1.1 eq.) and Et₃N (6.80 mL, 1.1 eq.) successively.

The mixture was refluxed for 20 h and then concentrated under vacuum, DCM was added and the mixture was concentrated again (2×). The brown oil resulting was dissolved in 100 ml of MeOH and H₂O was added dropwise. The solid precipitated was filtered, washed with H₂O and dried to afford 12.84 g of the desired compound as pale brown solid. Yield 90%. ¹H NMR (300 MHZ, DMSO-d₆) δ 10.79 (br. s., 1H), 8.03 (d, J=1.76 Hz, 1H), 7.54 (d, J=8.49 Hz, 1H), 7.38 (dd, J=1.90, 8.49 Hz, 1H), 6.75 (s, 1H), 1.48 (s, 9H). ¹³C NMR (101 MHZ, DMSO-d₆) δ 152.87, 143.01, 137.38, 136.29, 127.73, 124.52, 123.57, 114.65, 104.39, 81.11, 28.44. ESIMS m/z: other signals [M+H]⁺. Purity was determined as >95% by HPLC (327 nm), Rt: 3.94 min (Sunfire 3.5μ, C18 4.6×50 mm, ammonium acetate 0.05% pH 7/Acetonitrile).

### Intermediate 4: (6-bromo-1-benzothien-2-yl)amine hydrochloride

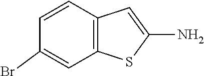 [SMILES: Nc1cc2ccc(Br)cc2s1]

To a solution of intermediate 3 (12.84 g, 39.12 mmol) in MeOH (250 mL, 6.4 mL/mmol) at 0° C. under nitrogen atmosphere was added dropwise acetyl chloride (14 mL, 5 eq.) (exothermic reaction). The mixture was allowed to warm to room temperature and was stirred overnight. Then, half of the solvent was removed under vacuum; the precipitated solid was filtered and washed with t-BuOMe. 8.24 g of the desired compound were obtained as pale brown solid. Yield 80%. ¹H NMR (300 MHZ, DMSO-d₆) δ 7.84-7.71 (m, 1H), 7.23 (d, J=1.03 Hz, 3H), 6.04 (s, 2H). ESIMS m/z: 228 [M+H]⁺. Purity was determined as >95% by HPLC (288 nm), Rt: 3.18 min (Waters Sunfire 3μ C18; 4.6×50 mm, formic acid 0.1% pH 2.4/Acetonitrile).

### Intermediate 5: 7-bromo-2-methylbenzo[4,5]thieno[2,3-b]pyridin-4(1H)-one

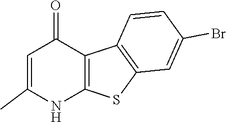 [SMILES: Cc1cc(=O)c2c([nH]1)sc1cc(Br)ccc12]

To a suspension of intermediate 4 (2.3 g, 8.7 mmol) in DCM (300 mL, 34 mL/mmol) was added 1 N NaOH (200 mL) and after some minutes 2 N NaOH (50 mL). The mixture was stirred to obtain two layers. Aqueous layer was washed with DCM, combined organic layers were dried over Na₂SO₄ and concentrated to dryness. The free amine obtained (1.98 g, 8.7 mmol) was solved with dry Toluene (60 mL, 7 mL/mmol) and transferred to a round bottom flask equipped with a Dean-Stark system. AcOH (1.6 mL, 5 eq.) and ethyl acetoacetate (1.76 mL, 1.5 eq.) were added and the mixture was refluxed until 30 mL of toluene were distilled, then reaction mixture was concentrated to dryness under vacuum. Over this crude was added diphenyl ether (20 mL, 2.3 mL/mmol) and the mixture was refluxed for 1 h. Reaction was allowed to warm to room temperature and then t-BuOMe (20 mL, 2.3 mL/mmol) was added. The precipitated solid was filtered and dried under vacuum to afford 1.27 g of the titled compound as yellow crystalline solid. Yield 55%. ¹H NMR (300 MHZ, DMSO-d₆) δ 11.88 (br. s., 1H), 8.40 (br. s., 1H), 8.27 (s, 1H), 7.63 (d, J=8.49 Hz, 1H), 6.73 (br. s., 1H), 2.45 (br. s., 3H). ESIMS m/z: 294 [M+H]⁺. Purity was determined as >95% by HPLC (254 nm), Rt: 2.66 min (Sunfire 3.5μ C18; 4.6×50 mm, ammonium acetate 0.05% pH 7/Acetonitrile).

### Intermediate 6: 7-bromo-3-chloro-2-methylbenzo[4,5]thieno[2,3-b]pyridin-4(1H)-one

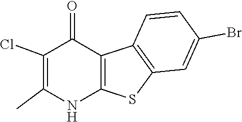 [SMILES: Cc1[nH]c2sc3cc(Br)ccc3c2c(=O)c1Cl]

To a solution of Intermediate 5 (400 mg, 1.36 mmol) in NMP (12 mL, 8.8 mL/mmol) at 0° C., TCCA (132 mg, 1.3 eq) was added under nitrogen atmosphere. The mixture was stirred at 0° C. for 2 h and then added over 1 N NH₄Cl dropwise. The precipitated solid was filtered, washed with 1 N NH₄Cl and H₂O and finally with ACN. The resulting solid was vigorously stirred with 5 mL of ACN for 2 h, filtered and washed again with 2 mL of ACN. 320 mg of the titled compound were obtained as pale brown solid. Yield 72%. ¹H NMR (300 MHZ, DMSO-d₆) δ 13.16 (br. s., 1H), 8.55 (br. s., 1H), 8.29 (br. s., 1H), 7.64 (br. s., 1H), 2.49 (s, 3H). ESIMS m/z: 328 [M+H]⁺. Purity was determined as >95% by HPLC (254 nm), Rt: 2.42 min (Ace C18; 3μ; 30×4.6 mm, formic acid 0.1% pH 2.4/Acetonitrile).

### Intermediate 7: 1-({4-[(trifluoromethyl)oxy]phenyl}methyl)-4-piperidinone

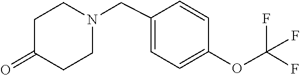 [SMILES: O=C1CCN(Cc2ccc(OC(F)(F)F)cc2)CC1]

To a solution of tert-butyl 4-oxo-1-piperidinecarboxylate (3 g, 1.5 mmol) in dry MeOH (20 mL, 13 mL/mmol) under N₂ atm, HCl (4M in dioxane) (20 mL, 5 eq.) was added. The mixture was stirred at room temperature overnight and then concentrated to dryness under vacuum. The resulting solid was suspended in ACN and stirred over 15 min, filtered and washed with ACN to afford 2 g of the titled compound as a white solid. Yield quantitative. This compound was suspended in dry DCM (50 mL, 35 mL/mmol) under N₂ atm, Et₃N (4.9 mL, 2.5 eq.) was added and the mixture was stirred at room temperature for 30 min. After that, 4-(trifluoromethoxy)benzyl bromide (3.4 mL, 1.5 eq.) was added. The resulting mixture was stirred at room temperature overnight and then, concentrated to dryness under vacuum and purified by column chromatography on silica gel, eluting with mixtures MeOH/DCM (0-10%) to afford 830 mg of the titled product. Yield 21%. ¹H NMR (300 MHZ, DMSO-d₆) δ 7.54-7.40 (m, 2H), 7.32 (d, J=7.91 Hz, 2H), 3.63 (s, 2H), 2.74-2.60 (m, 4H), 2.34 (t, J=6.08 Hz, 4H).

### Intermediate 8: N-methyl-1-({4-[(trifluoromethyl)oxy]phenyl}methyl)-4-piperidinamine

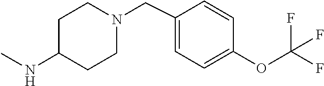 [SMILES: CNC1CCN(Cc2ccc(OC(F)(F)F)cc2)CC1]

Intermediate 7 was solved in DCE (10 mL, 7.6 mL/mmol) under N₂ atm, and methylamine hydrochloride (98 mg, 1.1 eq.) was added. The mixture was stirred for 1 h and then NaH(AcO)₃ (695 mg, 2.5 eq.) was added. The resulting mixture was stirred at room temperature overnight. Afterwards was concentrated to dryness under vacuum, dissolved in DCM and washed with sat NaHCO₃ by extraction. Organic layer was dried over Na₂SO₄, filtered and concentrated to give 333 mg of the title compound. Yield 88%. ¹H NMR (300 MHZ, DMSO-d₆) δ 7.43-7.34 (m, 2H), 7.33-7.18 (m, 2H), 3.44 (s, 2H), 2.70 (d, J=11.86 Hz, 2H), 2.23 (s, 3H), 1.94 (dt, J=2.12, 11.46 Hz, 2H), 1.74 (d, J=13.33 Hz, 2H), 1.29-1.12 (m, 2H).

### Intermediate 9: 7-(2-fluoro-4-(trifluoromethyl)phenyl)-2-methylbenzo[4,5]thieno[2,3-b]pyridin-4(1H)-one

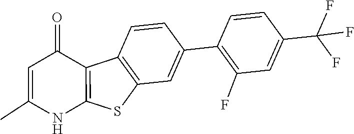 [SMILES: Cc1cc(=O)c2c([nH]1)sc1cc(-c3ccc(C(F)(F)F)cc3F)ccc12]

Scheme 3 route: A suspension of Intermediate 5 (200 mg, 0.68 mmol) in DME (3.4 mL, 5 mL/mmol) was added over the catalyst Pd(PPh₃)₄ (78 mg, 0.1 eq.) in a 25 mL round-bottomed flask under N₂ atmosphere. A solution of 2-fluoro-4-(trifluoromethyl)phenylboronic acid (212 mg, 1.5 eq.) in EtOH (1.7 mL, 2.5 mL/mmol) and a solution of Na₂CO₃ (575.8 mg, 8 eq.) in water (2.8 mL, 4.1 mL/mmol) were added successively. The reaction was heated at 110° C. for 5 h. The crude was cooled down and water was added. The precipitate obtained was then filtered and washed with more water, afterwards was purified by column chromatography on silica gel, eluting with mixtures MeOH/DCM (0-20%) to afford 100 mg of the desired compound as yellow solid. Yield 39%. ¹H NMR (0, ppm, DMSO-d₆) δ 11.83 (bs, 1H); 8.52 (bs, 1H); 8.24 (s, 1H); 7.85 (m, 2H); 7.72 (m, 2H); 6.80 (bs, 1H); 2.49 (s, 3H). ESIMS m/z: 378 [M+H]⁺. Purity was determined as >95% by HPLC (300 nm), Rt: 3.74 min (Waters Sunfire 3.5μ; C18; 3×30 mm, ammonium acetate 0.05% pH 7/Acetonitrile).

### Scheme 5 Route: Via Intermediates 12-14.

### Preparation of Intermediate 12

To a suspension of Intermediate 3 in 1,4-dioxane and water was added K₂CO₃. The reaction mixture was heated, then Pd(dppf)Cl₂ was added. A solution 2-fluoro-4-(trifluoromethyl)phenyl)boronic acid in 1,4-dioxane was added slowly. The reaction mixture was stirred until the reaction was complete. The mixture was cooled and MTBE and water were added. The layers were separated and the organic layer washed with water. The organic phase was heated and filtered through CUNO, and CUNO pad washed with MTBE. The filtrate was concentrated and switched to IPA. Water was added and the resulting suspension was filtered, and the cake washed with water. The wet cake was dried under vacuum to afford Intermediate 12.

### Preparation of Intermediate 13

A solution of HCl in MeOH was added to a suspension of Intermediate 12 in MeOH. The reaction mixture was stirred until the reaction was complete. The reaction mixture was concentrated, and solvent switched to EtOAc. The resultant mixture was cooled, filtered and the cake washed with EtOAc. The wet cake was dried under vacuum to afford Intermediate 13.

### Preparation of Intermediate 14

A mixture of K₂CO₃, process water and 2-MeTHF was cooled and Intermediate 13 added. The mixture was stirred and the layers separated. The organic layer was washed with aqueous Na₂SO₄ solution. The organic layer was concentrated under vacuum, and solvent switched to 2-MeTHF to give a 2-MeTHF solution of Intermediate 13 free base.

A mixture of cyclohexane, ethyl acetoacetate and AcOH was heated to reflux. The 2-MeTHF solution of Intermediate 13 free base was charged slowly. The reaction was stirred until complete. The reaction mixture was cooled, and diluted with 2-MeTHF. The resulting solution was added slowly to aqueous Na₂CO₃ solution. The mixture was stirred, and the layers separated. The organic layer was washed with water. The organic layer was concentrated, and solvent switched to 2-MeTHF to remove water, and then switched to NMP to obtain Intermediate 14 as a solution in NMP.

### Preparation of Intermediate 9

The NMP solution of Intermediate 14 was pumped through a tube reactor at high temperature. The reaction solution was cooled, and MeCN added. The mixture was seeded and stirred. MeCN was added. The suspension was cooled, filtered, and cake washed with MeCN. The cake was dried to give Intermediate 9.

### Intermediate 10: 7-(4-(hydroxymethyl)phenyl)-2-methylbenzo[4,5]thieno[2,3-b]pyridin-4(1H)-one

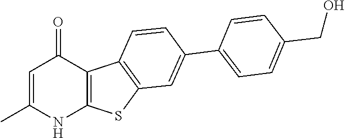 [SMILES: Cc1cc(=O)c2c([nH]1)sc1cc(-c3ccc(CO)cc3)ccc12]

A septum-sealed microwave tube (2-5 mL) was charged with Intermediate 5 (prepared as above; 150 mg, 0.5 mmol), Pd(PPh₃)₄ (30 mg, 0.05 eq.), Na₂CO₃ (108 mg, 2 eq.), 4-(hydroxymethyl) benzene boronic acid (77.5 mg, 1 eq.) and solved in a mixture DME/H₂O/EtOH (4:2:1) (5 mL, 10 mL/mmol). The mixture was heated at 250° C. (max Pressure 16 bar) under microwave radiation for 7 min. After cooling, water was added and the precipitate filtered and washed with more water. The crude was purified by column chromatography on silica gel, eluting with mixtures MeOH/DCM (0-10%) to afford 28 mg of the desired compound as a yellowish solid. Yield 17%. ¹H NMR (300 MHZ, DMSO-d₆) δ 12.02-11.40 (m, 1H), 8.64-8.38 (m, 1H), 8.28 (d, J=0.73 Hz, 1H), 7.81-7.75 (m, 1H), 7.73 (d, J=8.06 Hz, 2H), 7.42 (d, J=8.05 Hz, 2H), 6.89-6.65 (m, 1H), 5.24 (t, J=5.57 Hz, 1H), 4.55 (d, J=5.42 Hz, 2H). ESIMS m/z: 322 [M+H]⁺. Purity was determined as >95% by HPLC (230 nm), Rt: 1.76 min (Ace C18; 3μ; 30×4.6 mm, formic acid 0.1% pH 2.4/Acetonitrile).

### Intermediate 11: 4-(3-chloro-2-methyl-4-oxo-1,4-dihydrobenzo[4,5]thieno[2,3-b]pyridin-7-yl)benzaldehyde

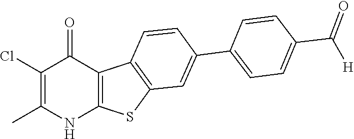 [SMILES: Cc1[nH]c2sc3cc(-c4ccc(C=O)cc4)ccc3c2c(=O)c1Cl]

A septum-sealed microwave tube (10-20 mL) was charged with Intermediate 6 (300 mg, 0.91 mmol) and DME (5.7 mL, 6.2 mL/mmol). Pd(PPh₃)₄ (53 mg, 0.05 eq.) and Na₂CO₃ (193 mg, 2 eq.) in H₂O (3 mL, 3.3 mL/mmol) and 4-formylphenylboronic acid (274 mg, 2 eq.) were added. The mixture was deoxygenated by bubbling of N₂ for some min and then, was heated at 250° C. (max Pressure 16 bar) under microwave radiation for 7 min. After cooling, water was added and the precipitate filtered and washed with more water. The resulting solid was triturated with DCM, stirred overnight and filtered washing with more DCM to afford 250 mg of the desired compound as a brown solid. Yield 77%. ¹H NMR (300 MHZ, DMSO-d₆) δ 13.38-12.76 (m, 1H), 10.06 (s, 1H), 8.75 (d, J=8.35 Hz, 1H), 8.45 (s, 1H), 8.01 (s, 4H), 7.91 (d, J=8.35 Hz, 1H), 2.52 (br. s., 3H).

### Example 1a: 3-chloro-7-(2-fluoro-4-(trifluoromethyl)phenyl)-2-methylbenzo[4,5] thieno[2,3-b]pyridin-4(1H)-one (Preparation Method 1)

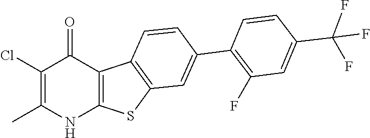 [SMILES: Cc1[nH]c2sc3cc(-c4ccc(C(F)(F)F)cc4F)ccc3c2c(=O)c1Cl]

This compound was prepared by chlorination of the intermediate 9 using TCCA by the same method described for intermediate 6 to lead a whitish solid. Yield 42%. ¹H NMR (300 MHZ, DMSO-d₆) δ 13.15 (br. s., 1H), 8.76 (d, J=8.20 Hz, 1H), 8.27 (s, 1H), 7.91-7.79 (m, 2H), 7.72 (dd, J=2.56, 7.69 Hz, 2H), 2.53 (s, 3H). ESIMS m/z: 410 [M−H]−. Purity was determined as >95% by HPLC (308 nm), Rt: 3.53 min (sunfire 3.5μ; C18; 4.6×50 mm, formic acid 0.1% pH 2.4/Acetonitrile).

### Example 1b: 3-chloro-7-(2-fluoro-4-(trifluoromethyl)phenyl)-2-methylbenzo[4,5] thieno[2,3-b]pyridin-4(1H)-one (Preparation Method 2)

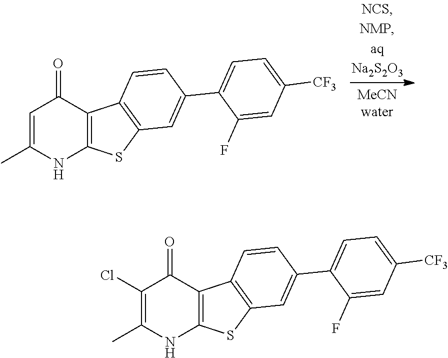 [SMILES: Cc1cc(=O)c2c([nH]1)sc1cc(-c3ccc(C(F)(F)F)cc3F)ccc12 ; Cc1[nH]c2sc3cc(-c4ccc(C(F)(F)F)cc4F)ccc3c2c(=O)c1Cl]

A 5 L flask with magnetic stirring was charged with Intermediate 9 (80 g). NMP (2000 mL, 25V) was added, under a N₂ atmosphere. NCS (28.3 g, 1.0 eq) was added in portions. The temperature was adjusted to 40-50° C., and the mixture was stirred at 40-50° C. for 16 h. A sample was taken for analysis. Charge another NCS (4.25 g, 0.15 eq) into R¹. Stir R¹ at 40-50° C. for another 3 h. Take sample for analysis. Adjust R1 to 15-25° C., charge 5% Na₂S₂O₃ (2000 mL, 25V) solution slowly into R¹ at 15-25° C. Stir R¹ at 15-25° C. for 1 h, and filter. The wet cake was washed with MeCN (160 mL, 2V) three times. The wet cake was washed with water (160 mL, 2V) three times to give crude 3-chloro-7-(2-fluoro-4-(trifluoromethyl)phenyl)-2-methylbenzo[4,5] thieno[2,3-b]pyridin-4(1H)-one.

### Example 1c(i): crystalline form of 3-chloro-7-(2-fluoro-4-(trifluoromethyl)phenyl)-2-methylbenzo[4,5] thieno[2,3-b]pyridin-4(1H)-one-Form 1

Crude 3-chloro-7-(2-fluoro-4-(trifluoromethyl)phenyl)-2-methylbenzo[4,5] thieno[2,3-b]pyridin-4(1H)-one was dissolved in NMP (2.4 L) at 95-105° C. The clear solution was cooled to 75-85° C. The resulting suspension was stirred at 75-85° C. for 2 h, cooled to 50-60° C. and stirred at 50-60° C. for 8 h. The suspension was cooled to 20-30° C. and water (1.2 L) was added drop wise at 20-30° C. The suspension was stirred at 20-30° C. for 1 h, filtered and washed sequentially with MeCN (400 mL×3) and water (400 mL×3). The solids were dried at 40-50° C. to afford 3-chloro-7-(2-fluoro-4-(trifluoromethyl)phenyl)-2-methylbenzo[4,5] thieno[2,3-b]pyridin-4(1H)-one (72 g) as a crystalline solid.

The X-ray powder diffraction (XRPD) pattern of this crystalline form is shown in FIG. 1, and a summary of the diffraction angles is given in Table 1 below. The XRPD analysis was conducted on a PANalytical X'Pert Pro powder diffractometer, model PW3040/60 using an X'Celerator detector. The acquisition conditions were: radiation: Cu Kα, generator tension: 40 kV, generator current: 45 mA, start angle: 2.0° 2θ, end angle: 40.0° 2θ, step size: 0.0167° 2θ, time per step: 31.75 seconds. The sample was prepared by mounting a few milligrams of sample on a silicon wafer (zero background plate), resulting in a thin layer of powder.

TABLE 1

| Diffraction | d- |
|---|---|
| angle (°2θ) | spacings (Å) |
| 5.2 | 16.9 |
| 8.6 | 10.3 |
| 10.5 | 8.5 |
| 12.5 | 7.1 |
| 13.5 | 6.5 |
| 15.6 | 5.7 |
| 18.8 | 4.7 |
| 19.7 | 4.5 |
| 20.9 | 4.2 |
| 21.9 | 4.0 |
| 26.2 | 3.4 |

### Example 1c(ii): crystalline form of 3-chloro-7-(2-fluoro-4-(trifluoromethyl)phenyl)-2-methylbenzo[4,5] thieno[2,3-b]pyridin-4(1H)-one-Form 2

Crude 3-chloro-7-(2-fluoro-4-(trifluoromethyl)phenyl)-2-methylbenzo[4,5] thieno[2,3-b]pyridin-4(1H)-one was dissolved in DMSO (95V) at 90-95° C. The clear solution was cooled to 75-80° C. within 3 hrs and seed added. The suspension was stirred at 75-80° C. for 2.5 hrs, and then cooled to 20-30° C. within 12 hrs. Water (15V) was added at 20-30° C. over 8.2 hrs, and the suspension was aged at 20-30° C. for 20 hrs. The suspension was filtered, washed with water and the wet cake dried under vacuum at 40-50° C. to afford 3-chloro-7-(2-fluoro-4-(trifluoromethyl)phenyl)-2-methylbenzo[4,5] thieno[2,3-b]pyridin-4(1H)-one as a crystalline solid.

The X-ray powder diffraction (XRPD) pattern of this crystalline form is shown in FIG. 2, and a summary of the diffraction angles is given in Table 2 below. The XRPD analysis was conducted on a PANalytical X'Pert Pro powder diffractometer, model PW3040/60 using an X'Celerator detector. The acquisition conditions were: radiation: Cu Kα, generator tension: 45 kV, generator current: 40 mA, start angle: 2.0° 2θ, end angle: 40.0° 2θ, step size: 0.0167° 2θ, time per step: 31.75 seconds. The sample was prepared by mounting a few milligrams of sample on a silicon wafer (zero background plate), resulting in a thin layer of powder.

TABLE 2

| Diffraction angle (°2θ) | d-spacings (Å) |
|---|---|
| 5.9 | 15.0 |
| 9.9 | 8.9 |
| 11.8 | 7.5 |
| 13.9 | 6.4 |
| 14.2 | 6.2 |
| 15.4 | 5.7 |
| 19.9 | 4.5 |
| 23.2 | 3.8 |
| 23.7 | 3.8 |
| 24.2 | 3.7 |
| 28.6 | 3.1 |
| 29.7 | 3.0 |
| 35.8 | 2.5 |
| 35.9 | 2.5 |

### Example 2: Sodium 3-chloro-7-[2-fluoro-4-(trifluoromethyl)phenyl]-2-methyl[1]benzothieno[2,3-b]pyridin-4-olate

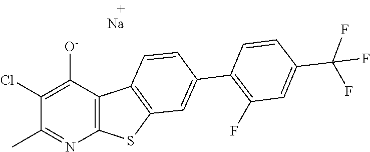

This compound was prepared by addition of one equivalent of aqueous sodium hydroxide to a suspension of the Example 1 in MeOH. The resulting solution was stirred for a few minutes and then concentrated under vacuum. ¹H NMR (300 MHZ, DMSO-d₆) δ 8.77 (d, J=8.20 Hz, 1H), 8.04 (s, 1H), 7.91-7.74 (m, 2H), 7.68 (d, J=8.06 Hz, 1H), 7.58 (td, J=1.59, 8.24 Hz, 1H), 2.42 (s, 3H). ESIMS m/z: 410 [M−H]−. Purity was determined as >95% by HPLC (334 nm), Rt: 3.52 min (sunfire 3.5μ; c18; 4.6×50 mm, formic acid 0.1% pH 2.4/Acetonitrile).

### Example 3: 3-chloro-7-(2-fluoro-5-(trifluoromethyl)phenyl)-2-methylbenzo[4,5]thieno[2,3-b]pyridin-4(1H)-one

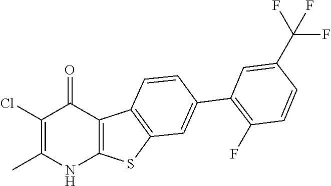 [SMILES: Cc1[nH]c2sc3cc(-c4cc(C(F)(F)F)ccc4F)ccc3c2c(=O)c1Cl]

This compound was prepared by a method analogous to that described for example 1 using the appropriate commercially available boronic acid and intermediate 6 as starting material. Yield 23%. ¹H NMR (300 MHZ, DMSO-d₆) δ 13.19 (s, 1H), 8.75 (d, J=8.49 Hz, 1H), 8.28 (s, 1H), 7.98 (d, J=6.59 Hz, 1H), 7.82 (br. s., 1H), 7.73 (d, J=8.20 Hz, 1H), 7.60 (t, J=9.30 Hz, 1H), 2.52 (s, 3H). ESIMS m/z: 412 [M+H]⁺; 410 [M−H]. Purity was determined as >95% by HPLC (305 nm), Rt: 3.45 min (Sunfire C18; 3.5μ; 50×4.6 mm, formic acid 0.1% pH 2.4/Acetonitrile).

### Example 4: 3-chloro-2-methyl-7-(2-(trifluoromethoxy)phenyl)benzo[4,5]thieno[2,3-b]pyridin-4(1H)-one

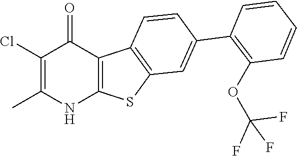 [SMILES: Cc1[nH]c2sc3cc(-c4ccccc4OC(F)(F)F)ccc3c2c(=O)c1Cl]

This compound was prepared by a method analogous to that described for example 1 using the appropriate commercially available boronic acid and intermediate 6 as starting material. Yield 29%. ¹H NMR (300 MHZ, DMSO-d₆) δ 13.14 (br. s., 2H), 8.72 (d, J=8.35 Hz, 1H), 8.12 (s, 1H), 7.56-7.66 (m, 2H), 7.55-7.45 (m, 3H), 2.53 (s, 3H). ESIMS m/z: 410 [M+H]⁺. Purity was determined as >90% by HPLC (254 nm), Rt: 3.38 min (Sunfire C18; 3.5μ; 50×4.6 mm, formic acid 0.1% pH 2.4/Acetonitrile).

### Example 5: 7-(2,4-bis(trifluoromethyl)phenyl)-3-chloro-2-methylbenzo[4,5]thieno[2,3-b]pyridin-4(1H)-one

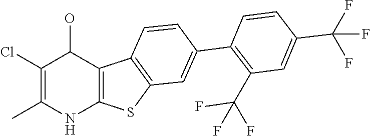 [SMILES: CC1=C(Cl)C(O)c2c(sc3cc(-c4ccc(C(F)(F)F)cc4C(F)(F)F)ccc23)N1]

This compound was prepared by a method analogous to that described for example 1 using the appropriate commercially available boronic acid and intermediate 46a as starting material. Yield 6%. ¹H NMR (300 MHZ, DMSO-d₆) δ 13.35-12.95 (m, 1H), 8.73 (d, J=8.20 Hz, 1H), 8.17 (br. s., 2H), 8.04 (s, 1H), 7.76 (d, J=7.62 Hz, 1H), 7.48 (d, J=8.06 Hz, 1H), 2.53 (s, 3H). ESIMS m/z: 462 [M+H]⁺. Purity was determined as >95% by HPLC (254 nm), Rt: 3.63 min (Sunfire C18; 3.5μ, 50×4.6 mm, formic acid 0.1% pH 2.4/Acetonitrile).

### Example 6: 3-chloro-7-(4-(hydroxymethyl)phenyl)-2-methylbenzo[4,5]thieno[2,3-b]pyridin-4(1H)-one

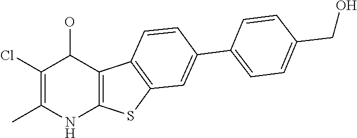 [SMILES: CC1=C(Cl)C(O)c2c(sc3cc(-c4ccc(CO)cc4)ccc23)N1]

To a solution of intermediate 10 (21 mg, 0.06 mmol) in NMP (1 mL, 16 mL/mmol), at 0° C. TCCA (6.5 mg, 1/3×1.3 eq) was added. The resulting solution was stirred at this temperature for 2 hours and then added over 1 N NH₄Cl solution. Precipitate was filtered, washed with water and then with ACN to afford 10 mg of the title compound as pale solid. Yield 43%. ¹H NMR (300 MHZ, DMSO-d₆) δ 13.34-12.92 (m, 1H), 8.71 (d, J=6.88 Hz, 1H), 8.32 (s, 1H), 7.81 (d, J=8.49 Hz, 1H), 7.72 (d, J=7.91 Hz, 2H), 7.42 (d, J=7.91 Hz, 2H), 5.36-5.09 (m, 1H), 4.55 (br. s., 2H). 2.52 (s, 3H). ESIMS m/z: 354 [M+H]⁺. Purity was determined as >95% by HPLC (254 nm), Rt: 2.04 min (Ace C18; 3μ; 30×4.6 mm, formic acid 0.1% pH 2.4/Acetonitrile).

### Example 7: 3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one 2,2,2-trifluoroacetate

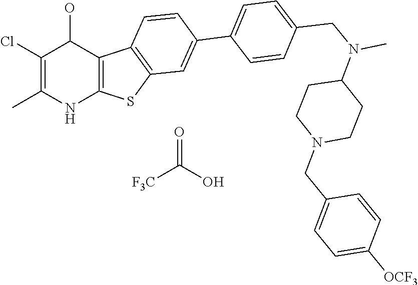 [SMILES: CC1=C(Cl)C(O)c2c(sc3cc(-c4ccc(CN(C)C5CCN(Cc6ccc(OC(F)(F)F)cc6)CC5)cc4)ccc23)N1 ; O=C(O)C(F)(F)F]

A septum-sealed microwave tube (0.5-2 mL) was charged with Intermediate 11 (73.4 mg, 0.208 mmol) and NMP (0.8 mL, 4 mL/mmol). Intermediate 8 (120 mg, 2 eq.) was added under N₂ atm and the mixture was stirred for some min. After that, NaBH(AcO)₃ (110 mg, 2.5 eq.) was added and the resulting mixture was heated at 140° C. (max. pressure of 16 bar) for 30 min. HPLC showed remaining starting material and some impurities, then NaHCO₃ was added and the mixture was extracted with DCM(×3). Combined organic layers were dried over Na₂CO₃, filtered and concentrated to dryness. The resulting yellow oil was dissolved in a bit of DCM and sat NH₄Cl was added. The solid wa7s filtered washing with more NH₄Cl and then purified by preparative HPLC eluting with mixtures ACN/H₂O (20-80% with 0.1% of TFA) to afford 10.7 mg of the desired compound as yellow solid. Yield 7%. ¹H NMR (300 MHZ, DMSO-d₆) δ 13.43-12.89 (m, 1H), 9.98 (br. s., 1H), 8.74 (d, J=8.20 Hz, 1H), 8.39 (d, J=1.32 Hz, 1H), 7.94-7.83 (m, 3H), 7.65 (d, J=8.05 Hz, 4H), 7.50 (d, J=8.20 Hz, 2H), 4.57-4.41 (m, 1H), 4.32 (br. s., 3H), 3.18-2.86 (m, 4H), 2.64 (br. s., 3H), 2.53 (s, 3H), 2.26 (d, J=1.90 Hz, 3H), 2.06-1.87 (m, 2H). ESIMS m/z: 625 [M+H]⁺. Purity was determined as >95% by HPLC (268 nm), Rt: 1.52 min (Sunfire C18, 3.5 m; 50×4.6 mm, formic acid 0.1% pH 2.4/Acetonitrile).

### Biological Data

The compounds of this invention may be tested in one of several biological assays to determine the concentration of the compound which is required to provide a given pharmacological effect. The assays are described below, with the results provided below in Table 2.

### Blood Stages Activity

The sensitivity of P. falciparum infected erythrocytes to the compound was determined using the [3H]hypoxanthine incorporation method with an inoculum of 0.5% parasitemia (ring stage) and 2% hematocrit. The parasites were grown in RPMI 1640, 25 mM HEPES and supplemented with 5% Albumax. Plates are incubated at 37° C., 5% CO₂, 5% O₂, 90% N₂. After 24 h of incubation, [3H]hypoxanthine is added and plates are incubated for another 24 h. After that period, plates are harvested on a glass fiber filter using a TOMTEC Cell harvester 96. Filters are dried and melt on scintillator sheets and the bound radioactivity is quantified by use of a Wallac Microbeta Trilux (Model 1450 LS-Perkin Elmer). IC50s are determined using Grafit 7 program (Grafit program; Erithacus Software, Horley, Surrey, United Kingdom).

### Enzymatic Bc1 Inhibition

For cytochrome bc₁ experiments, mitochondria were isolated as follows: parasitized erythrocytes were harvested by centrifugation and lysed with 0.05% (w/v) saponin in RPMI. Then parasites were washed three times with H-medium (0.07 M sucrose, 0.21 M mannitol, 1 mM EGTA, 5 mM MgCl₂, 5 mM KH₂PO₄, and 4 mM HEPES, pH 7.4) and resuspended in the same medium in the presence of 1 mM PMSF and protease inhibitor cocktail (Roche Complete). Parasites were disrupted by N₂ cavitation (4639 Cell disruption Bomb, Parr Instrument, Moline, IL, USA) at 1600 psi for 25 min at 4° C. Unbroken cells and cell debris were removed by centrifugation at 1,200×g for 10 min at 4° C. The mitochondrial fraction was pelleted at 10,000×g for 20 min at 4° C. The mitochondrial pellet was gently resuspended in 0.8 M sucrose, 1 mM EDTA, 10 mM Tris-HCl PH 7.4, and 0.1% BSA prior to separation on a 1-2 M sucrose gradient. The sample was centrifuged on a gradient at 80,000×g for 2 hr at 4° C. The mitochondria were then recovered and washed with 1 mM EDTA, 10 mM Tris-HCL pH 7.4 to remove sucrose, and resuspended in H-medium with protease inhibitor cocktail and 1 mM PMSF. Samples were stored at −80° C. until use. In the case of human mitochondria, HEK293 human cell line was used as starting material. Cells were pelleted at 400×g 10 min and disrupted by N₂ cavitation. Mitochondria then were isolated using the method described above. Cytochrome c reductase activity was assayed by a modification of the method of Fry and Pudney, Biochem. Pharmacol., 43 (1992), pp. 1545-1553. Mitochondria (40 μg/ml) were diluted in reaction buffer (250 mM sucrose, 50 mM KH₂PO₄, 0.2 mM EDTA, 1 mM NaN₃, and 2.5 mM KCN) containing 50 μM cytochrome c. Reactions were started by addition of 25 μM decylubiquinol and monitored by reduction of cytochrome c at 550 nm. To assure the linearity of the enzymatic reaction only data from the first 60 s were collected. Decylubiquinol substrate was prepared by reducing decylubiquinone (Sigma-Aldrich, St. Louis, MO, USA) in ethanol with sodium borohydride. Decylubiquinol was aliquoted and stored in acidified ethanol at −80° C. Inhibition of bc1 activity on mitochondria isolated from human parasites (P. falciparum 3D7A), rodent parasites (P. berghei ANKA) and human cells (HEK293).

Table 3: Biological data: Whole cell activity (WC IC50) need to be less than 1 μM, hbc1 IC50 is the activity in the human target (Higher than 5 μM, P.fbc1 IC50 is the activity in the P. falciparum target (less than 1 μM). Selectivity index to represent potential safety concerns, need to be higher than 100 folds.
<0.001 μM=++++
≥0.001 μM to <0.005 μM=+++
≥0.005 μM to <0.13 μM=++
>1 μM to <3 μM=+†††
>3 μM to <5 μM=††
>5 μM=†

TABLE 3

| Example | WC IC₅₀ | Hbc1 | p.fbc1 | Tox window |
|---|---|---|---|---|
| compound | (μM) | IC₅₀ (μM) | IC₅₀ (μM) | selectivity index |
| 1 | 0.001457 | 3.3 | 0.0046 | 300-600 |
| 3 | 0.031569 | 1.52 | 0.0018 | 500 |
| 4 | 0.000732 | 1.56 | 0.00096 | 1625 |
| 5 | 0.000433 | 2.42 | 0.0011 | 2200 |
| 6 | 0.000946 | 5.42 | | |
| 7 | 0.120841 | >10 | | |

### Biological Example 2

The biological activity of the compounds of the present application were also compared against a panel of P. falciparum strains with different genetic backgrounds, including multiple drug resistance to known antimalarials (chloroquine, pyrimethamine and/or atovaquone). Lack of cross-resistance was observed in most of the strains tested. A moderate degree of resistance was only observed when tested against Tm90C₂B which harbours the Y268S mutation in bc₁ which is highly resistant to atovaquone. The results are shown in Table 4.

TABLE 4

| | IC50 | | | | |
|---|---|---|---|---|---|
| | (mean μM ± | Pf IC50 Ratio vs Pf IC50 3D7 | | | |
| | SD) | | | | Tm90C |
| Compound | 3D7A | Dd2 | FCR3 | Tm90C2A | 2B |
| Example 1 | 0.012 ± 0.002 | 1 | 1 | 1 | 7 |
| Artesunate | 0.014 ± 0.0003 | 1 | 1 | 1 | 1 |
| Atovaquone | 0.001 ± 0.001 | 2 | >100 | 1 | >250 |
| Chloroquine | 0.029 ± 0.006 | >7 | >7 | >7 | >14 |
| Pyrimethamine | 0.032 ± 0.001 | >312 | 3 | >312 | >88 |
| Mefloquine | 0.037 ± 0.001 | 1 | 1 | 0.3 | 1 |
| Dihydroartemisinine | 0.004 ± 0.0001 | 1 | 1 | 1 | 1 |
| Lumefantrine | 0.011 ± 0.004 | 1 | 0 | 0.1 | 1 |

### Particle Size Determination

The sample vial was hand mixed by inverting it back and forth for about 30 seconds to ensure that there was no suspension sediment visible on the bottom of the vial. The suspension was added dropwise using a syringe, 1 ml tuberculin syringe with a 25 gauge needle (or equivalent). Specific gauge or size were not critical attributes. Sample was added until desired obscuration of 1%-5% was achieved. The dispersion was allowed to circulate in the Hydro MV for about 30 seconds. The sample measurement was performed using a Malvern MS3000 laser diffraction particle size analyzer to determine the particle size for the Formula (I) compounds using the following instrument parameters. No sonication was performed.

| Accessory Selection | |
|---|---|
| Accessory | Hydro MV |

Sample

| Particle Type | Non-Spherical |
|---|---|
| Refractive Index | 1.6 |
| Absorption Index | 0.01 |
| Refractive Index (blue) | 1.6 |
| Absorption Index (blue) | 0.01 |
| Density (g/cm3) | 1 |
| Dispersant Name | Water |
| Dispersant Refractive Index | 1.33 |
| Measurement | |
| Red Background Time (s) | 10 |
| Red Measurement Time (s) | 10 |
| Perform Blue Measurement | Yes |
| Blue Background Time (s) | 10 |
| Blue Measurement Time (s) | 10 |
| Number of Measurements | 3 |
| Delay between measurements | 10 |
| (s) | |
| Pre measurement delay (s) | 0 |
| Obscuration Limits (%) | 1.00-5.00 |
| Enable Autostart | No |
| Stabilisation time (s) | 30 |
| Enable Obscuration Filtering | No |
| Time-out (s) | 30 |
| Sample Dispersion | |
| Stir Speed (rpm) | 2000 |
| Tank Fill | Auto |
| Degas after Fill | Yes |
| Ultrasonic Mode | None |
| Clean Sequence Type | Normal |
| Clean Cycles | 3 |
| Sonicate During Clean | No |
| Data Processing | |
| Analysis Model | General Purpose |
| Keep a single result mode | No |
| Disable Detectors | 0 |
| Remove Blue Light | No |
| Analysis Sensitivity | Normal |
| Result Range (Microns) | 0.1-2100 |
| Distribution Type | Volume |

### Example Formulations for 3-chloro-7-(2-fluoro-4-(trifluoromethyl)phenyl)-2-methylbenzo[4,5] thieno[2,3-b]pyridin-4(1H)-one Form 1

For “Formulation 1”, Poloxamer P338-3% w/v; PEG 3350-3% w/v; Mannitol-3.5% w/v; Sterile water for injection-q.s. 100%; (2862 mg) was added to the vial containing 300 mg (>1 μm, micron) of 3-chloro-7-(2-fluoro-4-(trifluoromethyl)phenyl)-2-methylbenzo[4,5] thieno[2,3-b]pyridin-4(1H)-one. The mixture was vortexed for 2 minutes before a stir bar was added, and formulation was stirred overnight. Subsequentially, formulation was transferred into 5 mL centrifuge tube and using pipette QS to final volume of 3 mL to produce an opaque, white suspension at a target concentration of 100 mg/mL for intramuscular administration.

For “Formulation 2”, Poloxamer P338-3% w/v; PEG 3350-3% w/v; Mannitol-3.5% w/v; Sterile water for injection-q.s. 100%; (2862 mg) was added to the vial containing 300 mg (<1 μm, sub-micron) of 3-chloro-7-(2-fluoro-4-(trifluoromethyl)phenyl)-2-methylbenzo[4,5] thieno[2,3-b]pyridin-4(1H)-one. The mixture was vortexed for 2 minutes before a stir bar was added, and formulation was stirred overnight. Subsequentially, formulation was transferred into 5 mL centrifuge tube and using pipette QS to final volume of 3 mL to produce an opaque, white suspension at a target concentration of 100 mg/mL for intramuscular administration.

For “Formulation 3”, Poloxamer P338-1% w/v; PEG 3350-1% w/v; Mannitol-3.5% w/v; Sterile water for injection—q.s. 100%; (2862 mg) was added to the vial containing 300 mg (>1 μm, micron) of 3-chloro-7-(2-fluoro-4-(trifluoromethyl)phenyl)-2-methylbenzo[4,5] thieno[2,3-b]pyridin-4(1H)-one. The mixture was vortexed for 2 minutes before a stir bar was added, and formulation was stirred overnight. Subsequentially, formulation was transferred into 5 mL centrifuge tube and using pipette QS to final volume of 3 mL to produce an opaque, white suspension at a target concentration of 100 mg/mL for intramuscular administration.

### Procedure for Measuring Pharmacokinetic Parameters for “Formulation 1, 2, and 3” in an Intramuscular In Vivo Experiment

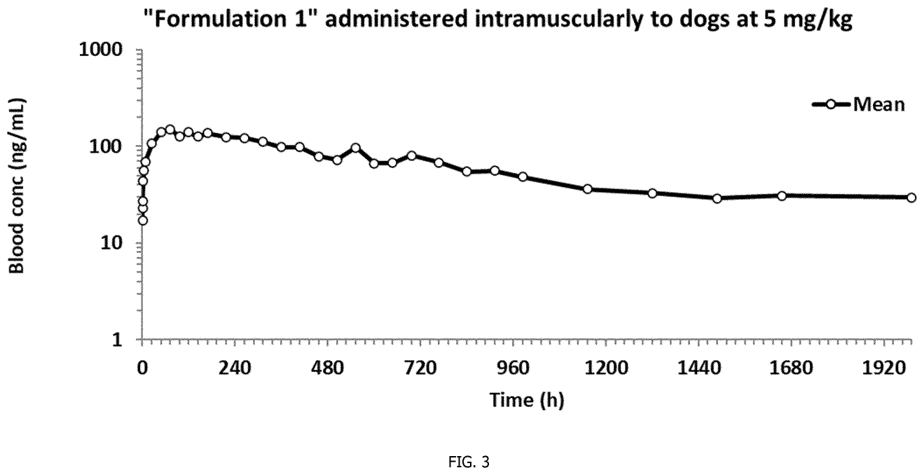

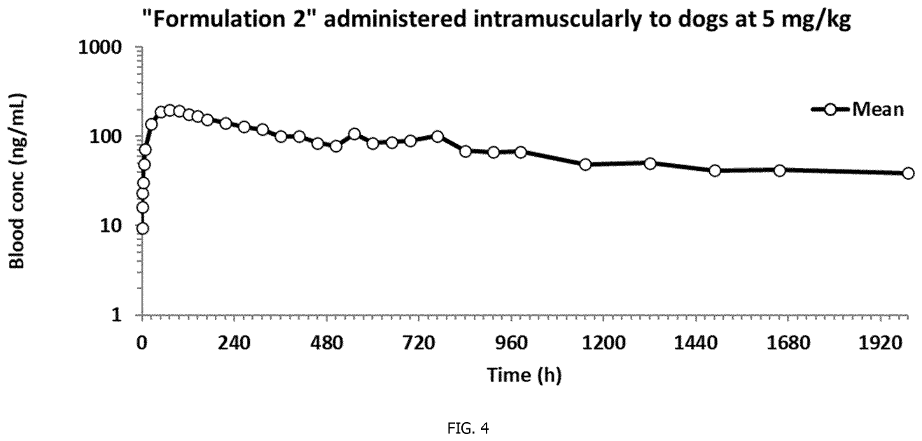

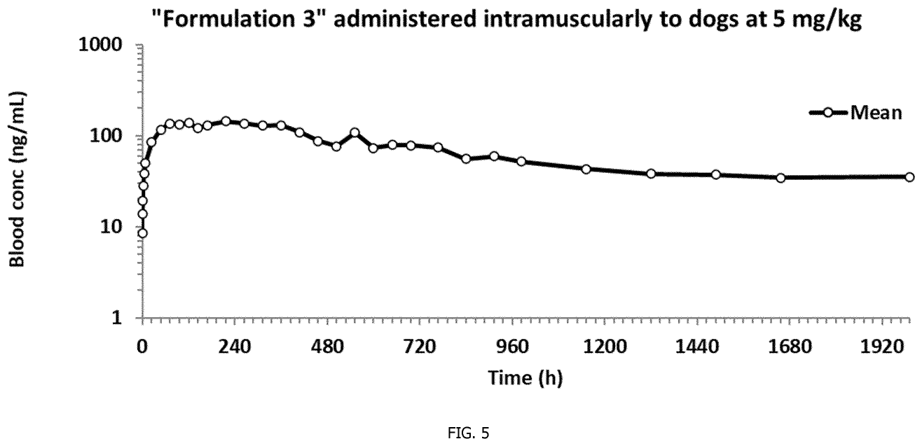

“Formulations 1, 2 and 3” of Form 1 were administered to Male Beagle Dogs as an intramuscular injection at a dose of 5 mg/kg. Blood samples were collected at hours 0.25 h, 0.5 h, 1 h, 2 h, 4 h, 8 h post-dose on Day 1, then subsequently on Days 2, 3, 4, 5, 6, 7, 8, 10, 12, 14, 16, 18, 20, 22, 24, 26, 28, 30, 33, 36, 39, 42, 49, 56, 63, 70 and 84. Blood samples were collected into tubes containing K₂EDTA anticoagulant, mixed by inversion, and maintained on wet ice until processing. As soon as possible, 100 μL aliquots of the blood sample were transferred into separate microtubes containing 100 μL of sterile water. Diluted blood samples were vortexed thoroughly and placed in dry ice until transferred to a freezer set to maintain −80° C. and protected from light until analysis by LC-MS/MS. All in vivo blood:water samples were injected on a Agilent 1200 series and detected using a MDS Sciex API 4500 or 5500 triple-quadrupole LC-MS/MS system. The analytical column used was an Agilent Zorbax SB-C₈ (30 mm×2.1 mm, 3.5 μm) maintained at room temperature. Mobile phase A consisted of 0.1% formic acid in 95:5 (v:v) water:acetonitrile. Mobile phase B consisted of 0.1% formic acid in 5:95 (v:v) iso-propyl-alcohol (IPA):acetonitrile. The flow rate was 0.8 ml/min. The gradient was as follows: Mobile B was held for 0.25 minutes at 55% and then linearly increased from 55% to 95% over 1 minute, maintained at 95% for 0.5 minute, then maintained at 55% for 0.67 minutes. Results of PK experiment are described in Tables 5, 6 and 7 and FIGS. 3, 4 and 5.

TABLE 5
Blood concentration vs. time data for a study evaluating “Form 1 Formulation 1”
administered to Beagle dogs intramuscularly at a dose of 5 mg/kg (n = 3)

| | | Dog 1001 | Dog 1002 | Dog 1003 | Mean Conc. | Std. Dev | |
|---|---|---|---|---|---|---|---|
| Days | Hours | (ng/ml) | (ng/ml) | (ng/ml) | (ng/ml) | (ng/ml) | % CV |
| 0.010 | 0.25 | 3.57 | 22.5 | 25.0 | 17.0 | 11.7 | 68.8% |
| 0.020 | 0.50 | 6.92 | 28.0 | 33.4 | 22.8 | 14.0 | 61.4% |
| 0.042 | 1.00 | 13.4 | 26.5 | 41.8 | 27.2 | 14.2 | 52.2% |
| 0.083 | 2.00 | 27.7 | 44.2 | 58.7 | 43.5 | 15.5 | 35.6% |
| 0.17 | 4.00 | 40.9 | 52.1 | 74.3 | 55.8 | 17.0 | 30.5% |
| 0.33 | 8.00 | 55.0 | 59.2 | 91.6 | 68.6 | 20.0 | 29.2% |
| 2.0 | 24 | 94.9 | 86.5 | 140 | 107 | 28.8 | 26.9% |
| 3.0 | 48 | 157 | 107 | 154 | 139 | 28.0 | 20.1% |
| 4.0 | 72 | 147 | 127 | 178 | 151 | 25.7 | 17.1% |
| 5.0 | 96 | 114 | 109 | 156 | 126 | 25.8 | 20.4% |
| 6.0 | 120 | 124 | 126 | 168 | 139 | 24.8 | 17.8% |
| 7.0 | 144 | 116 | 106 | 155 | 126 | 25.9 | 20.6% |
| 8.0 | 168 | 120 | 124 | 167 | 137 | 26.1 | 19.0% |
| 10 | 216 | 113 | 109 | 151 | 124 | 23.2 | 18.6% |
| 12 | 264 | 93.4 | 118 | 156 | 122 | 31.5 | 25.8% |
| 14 | 312 | 87.0 | 94.7 | 150 | 111 | 34.4 | 31.1% |
| 16 | 360 | 81.6 | 88.8 | 123 | 97.8 | 22.1 | 22.6% |
| 18 | 408 | 71.7 | 88.0 | 135 | 98.2 | 32.9 | 33.5% |
| 20 | 456 | 53.1 | 83.2 | 101 | 79.1 | 24.2 | 30.6% |
| 22 | 504 | 68.9 | 61.9 | 86.5 | 72.4 | 12.7 | 17.5% |
| 24 | 552 | 73.8 | 92.3 | 124 | 96.7 | 25.4 | 26.3% |
| 26 | 600 | 55.0 | 57.3 | 88.5 | 66.9 | 18.7 | 28.0% |
| 28 | 648 | 64.6 | 61.4 | 77.1 | 67.7 | 8.30 | 12.3% |
| 30 | 696 | 75.9 | 74.9 | 89.9 | 80.2 | 8.39 | 10.5% |
| 33 | 768 | 61.5 | 73.1 | 70.2 | 68.3 | 6.04 | 8.8% |
| 36 | 840 | 55.5 | 47.2 | 61.9 | 54.9 | 7.37 | 13.4% |
| 39 | 912 | 50.5 | 54.2 | 62.8 | 55.8 | 6.31 | 11.3% |
| 42 | 984 | 41.6 | 49.6 | 54.2 | 48.5 | 6.38 | 13.2% |
| 49 | 1152 | 28.8 | 40.0 | 39.0 | 35.9 | 6.20 | 17.2% |
| 56 | 1320 | 25.3 | 31.9 | 41.4 | 32.9 | 8.09 | 24.6% |
| 63 | 1488 | 26.1 | 26.3 | 34.4 | 28.9 | 4.74 | 16.4% |
| 70 | 1656 | 36.5 | 23.5 | 32.7 | 30.9 | 6.68 | 21.6% |
| 84 | 1992 | 27.4 | 29.8 | 31.9 | 29.7 | 2.25 | 7.6% |

TABLE 6
Blood concentration vs. time data for a study evaluating “Form 1 Formulation 2”
administered to Beagle dogs intramuscularly at a dose of 5 mg/kg (n = 3)

| | | Dog 2001 | Dog 2002 | Dog 2003 | Mean Conc. | Std. Dev | |
|---|---|---|---|---|---|---|---|
| Days | Hours | (ng/ml) | (ng/ml) | (ng/mL) | (ng/ml) | (ng/mL) | % CV |
| 0.010 | 0.25 | 2.95 | 22.8 | 2.74 | 9.50 | 11.5 | 121.3% |
| 0.020 | 0.50 | 15.3 | 28.1 | 5.82 | 16.4 | 11.2 | 68.2% |
| 0.042 | 1.00 | 25.5 | 36.2 | 7.74 | 23.1 | 14.4 | 62.1% |
| 0.083 | 2.00 | 30.6 | 45.4 | 15.0 | 30.3 | 15.2 | 50.1% |
| 0.17 | 4.00 | 47.3 | 70.2 | 29.2 | 48.9 | 20.5 | 42.0% |
| 0.33 | 8.00 | 74.2 | 98.4 | 43.8 | 72.1 | 27.4 | 37.9% |
| 2.0 | 24 | 134 | 172 | 108 | 138 | 32.2 | 23.3% |
| 3.0 | 48 | 201 | 231 | 144 | 192 | 44.2 | 23.0% |
| 4.0 | 72 | 217 | 230 | 152 | 200 | 41.8 | 20.9% |
| 5.0 | 96 | 203 | 216 | 163 | 194 | 27.6 | 14.2% |
| 6.0 | 120 | 208 | 192 | 131 | 177 | 40.6 | 23.0% |
| 7.0 | 144 | 184 | 189 | 140 | 171 | 27.0 | 15.8% |
| 8.0 | 168 | 181 | 164 | 125 | 157 | 28.7 | 18.3% |
| 10 | 216 | 174 | 134 | 118 | 142 | 28.8 | 20.3% |
| 12 | 264 | 153 | 118 | 117 | 129 | 20.5 | 15.9% |
| 14 | 312 | 161 | 89.1 | 110 | 120 | 37.0 | 30.8% |
| 16 | 360 | 127 | 78.6 | 96.7 | 101 | 24.5 | 24.3% |
| 18 | 408 | 114 | 82.9 | 106 | 101 | 16.1 | 16.0% |
| 20 | 456 | 99.3 | 68.7 | 87.2 | 85.1 | 15.4 | 18.1% |
| 22 | 504 | 86.3 | 61.4 | 88.8 | 78.8 | 15.1 | 19.2% |
| 24 | 552 | 108 | 97.8 | 116 | 107 | 9.12 | 8.5% |
| 26 | 600 | 88.2 | 72.4 | 95.1 | 85.2 | 11.6 | 13.7% |
| 28 | 648 | 98.3 | 69.9 | 91.9 | 86.7 | 14.9 | 17.2% |
| 30 | 696 | 109 | 60.3 | 99.6 | 89.6 | 25.8 | 28.8% |
| 33 | 768 | 103 | 75.6 | 126 | 102 | 25.2 | 24.9% |
| 36 | 840 | 68.2 | 54.6 | 84.5 | 69.1 | 15.0 | 21.7% |
| 39 | 912 | 73.5 | 57.3 | 70.7 | 67.2 | 8.66 | 12.9% |
| 42 | 984 | 81.9 | 46.7 | 74.8 | 67.8 | 18.6 | 27.5% |
| 49 | 1152 | 56.6 | 37.8 | 52.0 | 48.8 | 9.80 | 20.1% |
| 56 | 1320 | 55.5 | 37.8 | 57.9 | 50.4 | 11.0 | 21.8% |
| 63 | 1488 | 41.4 | 38.3 | 45.6 | 41.8 | 3.66 | 8.8% |
| 70 | 1656 | 42.4 | 34.1 | 49.7 | 42.1 | 7.81 | 18.6% |
| 84 | 1992 | 44.7 | 35.4 | 37.0 | 39.0 | 4.97 | 12.7% |

TABLE 7
Blood concentration vs. time data for a study evaluating “Form 1 Formulation 3”
administered to Beagle dogs intramuscularly at a dose of 5 mg/kg (n = 3)

| | | Dog 3001 | Dog 3002 | Dog 3003 | Mean Conc. | Std. Dev | |
|---|---|---|---|---|---|---|---|
| Days | Hours | (ng/ml) | (ng/ml) | (ng/ml) | (ng/ml) | (ng/ml) | % CV |
| 0.010 | 0.25 | 12.2 | 8.9 | 4.3 | 8.45 | 3.99 | 47.3% |
| 0.020 | 0.50 | 19.6 | 13.3 | 9.1 | 14.0 | 5.27 | 37.6% |
| 0.042 | 1.00 | 24.3 | 23.3 | 10.4 | 19.3 | 7.75 | 40.1% |
| 0.083 | 2.00 | 33.5 | 25.4 | 25.8 | 28.2 | 4.57 | 16.2% |
| 0.17 | 4.00 | 46.4 | 36.2 | 32.9 | 38.5 | 7.04 | 18.3% |
| 0.33 | 8.00 | 61.1 | 44.7 | 45.3 | 50.4 | 9.30 | 18.5% |
| 2.0 | 24 | 101 | 74.5 | 79.0 | 84.8 | 14.2 | 16.7% |
| 3.0 | 48 | 124 | 102 | 123 | 116 | 12.4 | 10.7% |
| 4.0 | 72 | 149 | 122 | 139 | 137 | 13.7 | 10.0% |
| 5.0 | 96 | 151 | 124 | 127 | 134 | 14.8 | 11.0% |
| 6.0 | 120 | 142 | 138 | 134 | 138 | 4.00 | 2.9% |
| 7.0 | 144 | 130 | 130 | 104 | 121 | 15.0 | 12.4% |
| 8.0 | 168 | 155 | 113 | 122 | 130 | 22.1 | 17.0% |
| 10 | 216 | 167 | 155 | 111 | 144 | 29.5 | 20.4% |
| 12 | 264 | 159 | 137 | 113 | 136 | 23.0 | 16.9% |
| 14 | 312 | 146 | 127 | 112 | 128 | 17.0 | 13.3% |
| 16 | 360 | 131 | 165 | 93.7 | 130 | 35.7 | 27.5% |
| 18 | 408 | 125 | 113 | 91.4 | 110 | 17.0 | 15.5% |
| 20 | 456 | 99.5 | 88.0 | 74.2 | 87.2 | 12.7 | 14.5% |
| 22 | 504 | 72.9 | 81.2 | 75.6 | 76.6 | 4.23 | 5.5% |
| 24 | 552 | 136 | 107 | 82.6 | 109 | 26.7 | 24.6% |
| 26 | 600 | 81.0 | 74.6 | 64.8 | 73.5 | 8.16 | 11.1% |
| 28 | 648 | 81.9 | 81.9 | 74.7 | 79.5 | 4.16 | 5.2% |
| 30 | 696 | 83.8 | 83.0 | 67.9 | 78.2 | 8.96 | 11.5% |
| 33 | 768 | 79.9 | 78.0 | 65.3 | 74.4 | 7.94 | 10.7% |
| 36 | 840 | 61.8 | 54.7 | 51.1 | 55.9 | 5.44 | 9.7% |
| 39 | 912 | 61.4 | 57.6 | 58.8 | 59.3 | 1.94 | 3.3% |
| 42 | 984 | 48.5 | 48.9 | 59.0 | 52.1 | 5.95 | 11.4% |
| 49 | 1152 | 53.9 | 34.1 | 42.0 | 43.3 | 9.97 | 23.0% |
| 56 | 1320 | 41.8 | 37.9 | 34.3 | 38.0 | 3.75 | 9.9% |
| 63 | 1488 | 40.9 | 36.8 | 34.0 | 37.2 | 3.47 | 9.3% |
| 70 | 1656 | 34.7 | 34.6 | 34.4 | 34.6 | 0.153 | 0.4% |
| 84 | 1992 | 41.5 | 34.9 | 29.9 | 35.4 | 5.82 | 16.4% |

### Example Formulations for Form 2 3-chloro-7-(2-fluoro-4-(trifluoromethyl)phenyl)-2-methylbenzo[4,5] thieno[2,3-b]pyridin-4(1H)-one

For “Formulation 4”, 4.5% Poloxamer P338-3% PEG3350-2% Mannitol (1.88 mL) was added to the vial containing 1.12 mL of (<1 μm, sub-micron) 3-chloro-7-(2-fluoro-4-(trifluoromethyl)phenyl)-2-methylbenzo[4,5] thieno[2,3-b]pyridin-4(1H)-one, at 267.6 mg/mL. The formulation was mixed to produce an opaque, white suspension at a target concentration of 100 mg/mL with final batch size of 3.0 mL for intramuscular administration

For “Formulation 5”, 4.5% Poloxamer P338-1.5% Polysorbate 20-2% Mannitol (1.92 mL) was added to the vial containing 1.12 mL of (<1 μm, sub-micron) 3-chloro-7-(2-fluoro-4-(trifluoromethyl)phenyl)-2-methylbenzo[4,5] thieno[2,3-b]pyridin-4(1H)-one, at 276.8 mg/ml. The formulation was mixed to produce an opaque, white suspension at a target concentration of 100 mg/mL with final batch size of 3.0 mL for intramuscular administration

For “Formulation 6”, 4.5% Poloxamer P338-0.66% CMC-2% Mannitol (1.92 mL) was added to the vial containing 1.12 mL of (<1 μm, sub-micron) 3-chloro-7-(2-fluoro-4-(trifluoromethyl)phenyl)-2-methylbenzo[4,5] thieno[2,3-b]pyridin-4(1H)-one, at 277.0 mg/mL. The formulation was mixed to produce an opaque, white suspension at a target concentration of 100 mg/mL with final batch size of 3.0 mL for intramuscular administration

### Procedure for Measuring Pharmacokinetic Parameters for “Form 2 Formulation 4, 5 and 6” in an Intramuscular In Vivo Experiment

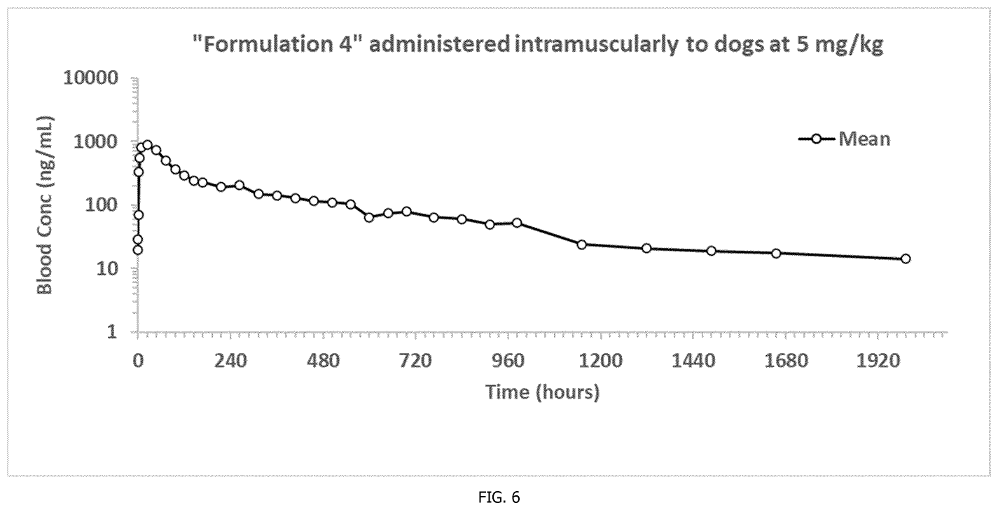

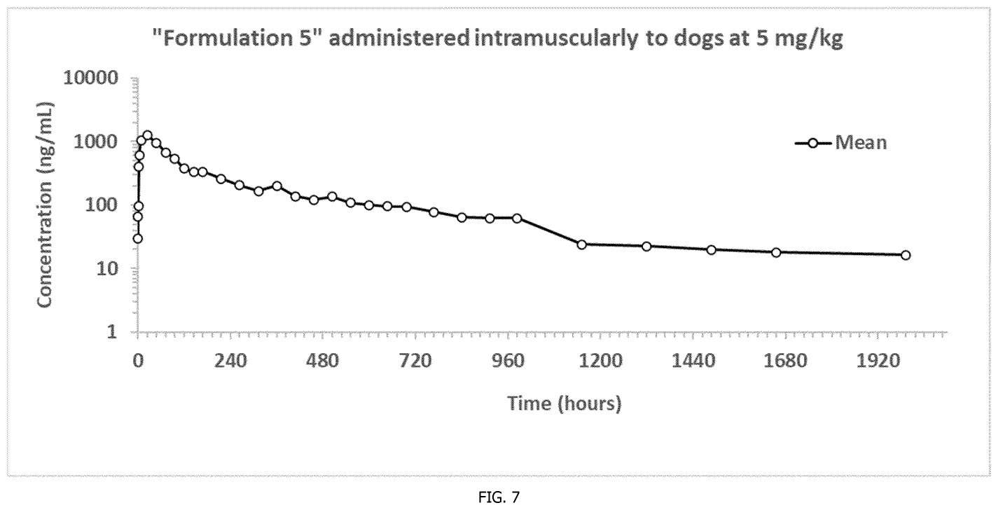

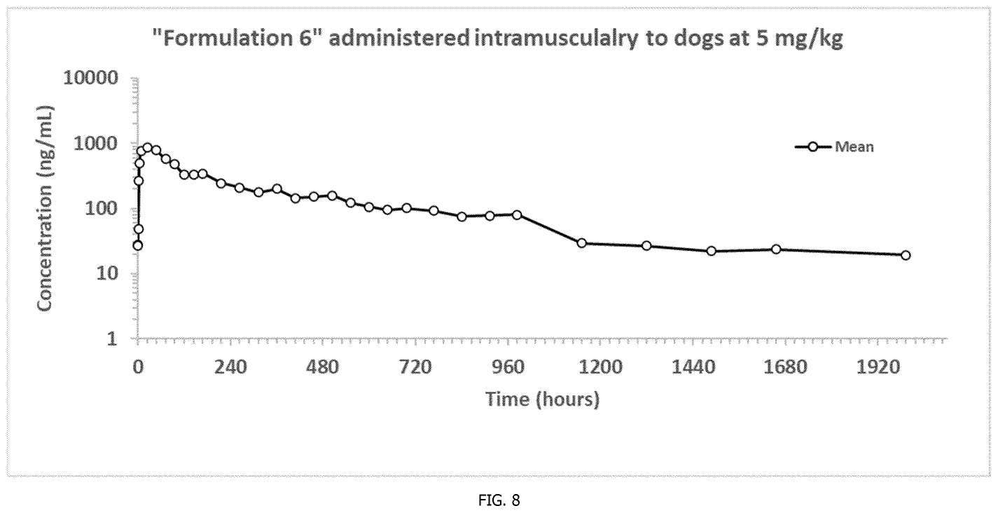

“Formulations 4, 5 and 6” of Form 2 were administered to Male Beagle Dogs as an intramuscular injection at a dose of 5 mg/kg. Blood samples were collected at hours 0.25 h, 0.5 h, 1 h, 2 h, 4 h, 8 h post-dose on Day 1, then subsequently on Days 2, 3, 4, 5, 6, 7, 8, 10, 12, 14, 16, 18, 20, 22, 24, 26, 28, 30, 33, 36, 39, 42, 49, 56, 63, 70 and 84. Blood samples were collected into tubes containing K₂EDTA anticoagulant, mixed by inversion, and maintained on wet ice until processing. As soon as possible, 100 μL aliquots of the blood sample were transferred into separate microtubes containing 100 μL of sterile water. Diluted blood samples were vortexed thoroughly and placed in dry ice until transferred to a freezer set to maintain −80° C. and protected from light until analysis by LC-MS/MS. All in vivo blood:water samples were injected on a Agilent 1200 series and detected using a MDS Sciex API 5500 triple-quadrupole LC-MS/MS system. The analytical column used was an Agilent Zorbax SB-C₈ (30 mm×2.1 mm, 3.5 μm) maintained at room temperature. Mobile phase A consisted of 0.1% formic acid in 95:5 (v:v) water:acetonitrile. Mobile phase B consisted of 0.1% formic acid in 50:50 (v:v) methanol:acetonitrile. The flow rate was 0.8 mL/min. The gradient was as follows: Mobile B was held for 0.25 minutes at 55% and then linearly increased from 55% to 70% over 1 minute, then increased to 95% over 0.08 minutes, maintained at 95% for 0.5 minute, then maintained at 55% for 0.67 minutes. Results of PK experiment are described in Tables 8, 9 and 10 and FIGS. 6, 7 and 8.

TABLE 8
Blood concentration vs. time data for a study evaluating “Formulation 4” administered
to Beagle dogs intramuscularly at a dose of 5 mg/kg (n = 3)

| | | Dog 1001 | Dog 1002 | Dog 1003 | Mean Conc. | Std. Dev | |
|---|---|---|---|---|---|---|---|
| Days | Hours | (ng/ml) | (ng/ml) | (ng/ml) | (ng/ml) | (ng/ml) | % CV |
| 0.010 | 0.25 | 17.8 | 12.8 | 29.0 | 19.9 | 8.30 | 41.8 |
| 0.020 | 0.50 | 23.5 | 24.8 | 38.3 | 28.9 | 8.20 | 28.4 |
| 0.042 | 1.00 | 33.4 | 30.1 | 144 | 69.2 | 64.8 | 93.7 |
| 0.083 | 2.00 | 380 | 148 | 480 | 336 | 170 | 50.7 |
| 0.17 | 4.00 | 728 | 273 | 631 | 544 | 240 | 44.1 |
| 0.33 | 8.00 | 1080 | 429 | 878 | 796 | 333 | 41.9 |
| 2.0 | 24 | 1110 | 641 | 882 | 878 | 235 | 26.7 |
| 3.0 | 48 | 823 | 699 | 646 | 723 | 90.8 | 12.6 |
| 4.0 | 72 | 546 | 553 | 408 | 502 | 81.8 | 16.3 |
| 5.0 | 96 | 396 | 364 | 334 | 365 | 31.0 | 8.5 |
| 6.0 | 120 | 270 | 314 | 299 | 294 | 22.4 | 7.6 |
| 7.0 | 144 | 222 | 271 | 234 | 242 | 25.5 | 10.5 |
| 8.0 | 168 | 228 | 256 | 197 | 227 | 29.5 | 13.0 |
| 10 | 216 | 166 | 225 | 184 | 192 | 30.2 | 15.8 |
| 12 | 264 | 134 | 309 | 168 | 204 | 92.8 | 45.6 |
| 14 | 312 | 119 | 188 | 139 | 149 | 35.5 | 23.9 |
| 16 | 360 | 120 | 170 | 133 | 141 | 25.9 | 18.4 |
| 18 | 408 | 103 | 165 | 116 | 128 | 32.7 | 25.5 |
| 20 | 456 | 98.2 | 140 | 107 | 115 | 22.0 | 19.2 |
| 22 | 504 | 83.4 | 141 | 107 | 110 | 29.0 | 26.2 |
| 24 | 552 | 86.0 | 124 | 98.5 | 103 | 19.4 | 18.8 |
| 26 | 600 | 60.1 | 76.0 | 55.8 | 64.0 | 10.6 | 16.6 |
| 28 | 648 | 49.5 | 95.2 | 77.5 | 74.1 | 23.0 | 31.1 |
| 30 | 696 | 64.8 | 89.6 | 80.4 | 78.3 | 12.5 | 16.0 |
| 33 | 768 | 47.4 | 87.0 | 58.9 | 64.4 | 20.4 | 31.6 |
| 36 | 840 | 44.6 | 85.9 | 51.3 | 60.6 | 22.2 | 36.6 |
| 39 | 912 | 50.9 | 55.3 | 42.8 | 49.7 | 6.34 | 12.8 |
| 42 | 984 | 40.3 | 70.2 | 46.6 | 52.4 | 15.8 | 30.1 |
| 49 | 1152 | 18.1 | 34.6 | 19.3 | 24.0 | 9.20 | 38.3 |
| 56 | 1320 | 15.4 | 27.8 | 18.8 | 20.7 | 6.41 | 31.0 |
| 63 | 1488 | 11.8 | 26.7 | 17.8 | 18.8 | 7.50 | 39.9 |
| 70 | 1656 | 12.3 | 26.1 | 14.2 | 17.5 | 7.48 | 42.7 |
| 84 | 1992 | 9.84 | 17.6 | 15.2 | 14.2 | 3.97 | 28.0 |

TABLE 9
Blood concentration vs. time data for a study evaluating “Formulation 5” administered
to Beagle dogs intramuscularly at a dose of 5 mg/kg (n = 3)

| | | Dog 2001 | Dog 2002 | Dog 2003 | Mean Conc. | Std. Dev | |
|---|---|---|---|---|---|---|---|
| Days | Hours | (ng/ml) | (ng/ml) | (ng/ml) | (ng/ml) | (ng/ml) | % CV |
| 0.010 | 0.25 | 31.3 | 28.1 | 30.0 | 29.8 | 1.61 | 5.4 |
| 0.020 | 0.50 | 56.8 | 51.4 | 89.8 | 66.0 | 20.8 | 31.5 |
| 0.042 | 1.00 | 65.3 | 65.9 | 154 | 95.1 | 51.0 | 53.7 |
| 0.083 | 2.00 | 233 | 376 | 609 | 406 | 190 | 46.7 |
| 0.17 | 4.00 | 426 | 480 | 943 | 616 | 284 | 46.1 |
| 0.33 | 8.00 | 781 | 505 | 1850 | 1045 | 710 | 68.0 |
| 2.0 | 24 | 1130 | 819 | 1830 | 1260 | 518 | 41.1 |
| 3.0 | 48 | 865 | 630 | 1370 | 955 | 378 | 39.6 |
| 4.0 | 72 | 600 | 473 | 941 | 671 | 242 | 36.1 |
| 5.0 | 96 | 603 | 354 | 665 | 541 | 165 | 30.4 |
| 6.0 | 120 | 449 | 248 | 425 | 374 | 110 | 29.4 |
| 7.0 | 144 | 382 | 217 | 400 | 333 | 101 | 30.3 |
| 8.0 | 168 | 420 | 214 | 376 | 337 | 108 | 32.2 |
| 10 | 216 | 338 | 186 | 255 | 260 | 76.1 | 29.3 |
| 12 | 264 | 257 | 152 | 209 | 206 | 52.6 | 25.5 |
| 14 | 312 | 215 | 131 | 152 | 166 | 43.7 | 26.3 |
| 16 | 360 | 288 | 150 | 171 | 203 | 74.4 | 36.6 |
| 18 | 408 | 177 | 107 | 130 | 138 | 35.7 | 25.9 |
| 20 | 456 | 141 | 96.7 | 124 | 121 | 22.3 | 18.5 |
| 22 | 504 | 169 | 105 | 134 | 136 | 32.0 | 23.6 |
| 24 | 552 | 134 | 84.4 | 108 | 109 | 24.8 | 22.8 |
| 26 | 600 | 120 | 74.1 | 107 | 100 | 23.7 | 23.6 |
| 28 | 648 | 118 | 72.1 | 95.2 | 95.1 | 23.0 | 24.1 |
| 30 | 696 | 115 | 83.6 | 84.0 | 94.2 | 18.0 | 19.1 |
| 33 | 768 | 97.0 | 56.3 | 79.3 | 77.5 | 20.4 | 26.3 |
| 36 | 840 | 73.1 | 59.7 | 59.2 | 64.0 | 7.88 | 12.3 |
| 39 | 912 | 78.2 | 53.0 | 54.4 | 61.9 | 14.2 | 22.9 |
| 42 | 984 | 74.3 | 51.3 | 61.6 | 62.4 | 11.5 | 18.5 |
| 49 | 1152 | 28.9 | 24.7 | 18.2 | 23.9 | 5.39 | 22.5 |
| 56 | 1320 | 25.3 | 21.3 | 20.8 | 22.5 | 2.47 | 11.0 |
| 63 | 1488 | 22.9 | 20.7 | 16.1 | 19.9 | 3.47 | 17.4 |
| 70 | 1656 | 21.3 | 18.2 | 14.5 | 18.0 | 3.40 | 18.9 |
| 84 | 1992 | 17.7 | 18.2 | 13.3 | 16.4 | 2.70 | 16.4 |

TABLE 10
Blood concentration vs. time data for a study evaluating “Formulation 6” administered
to Beagle dogs intramuscularly at a dose of 5 mg/kg (n = 3)

| | | Dog 3001 | Dog 3002 | Dog 3003 | Mean Conc. | Std. Dev | |
|---|---|---|---|---|---|---|---|
| Days | Hours | (ng/ml) | (ng/ml) | (ng/ml) | (ng/ml) | (ng/ml) | % CV |
| 0.010 | 0.25 | 17.6 | 44.4 | 22.1 | 28.0 | 14.4 | 51.2 |
| 0.020 | 0.50 | 24.6 | 34.6 | 20.9 | 26.7 | 7.09 | 26.5 |
| 0.042 | 1.00 | 28.5 | 92.2 | 22.4 | 47.7 | 38.7 | 81.0 |
| 0.083 | 2.00 | 158 | 421 | 210 | 263 | 139 | 53.0 |
| 0.17 | 4.00 | 250 | 744 | 500 | 498 | 247 | 49.6 |
| 0.33 | 8.00 | 423 | 1030 | 805 | 753 | 307 | 40.8 |
| 2.0 | 24 | 688 | 1040 | 821 | 850 | 178 | 20.9 |
| 3.0 | 48 | 672 | 1010 | 674 | 785 | 195 | 24.8 |
| 4.0 | 72 | 486 | 797 | 439 | 574 | 195 | 33.9 |
| 5.0 | 96 | 431 | 603 | 385 | 473 | 115 | 24.3 |
| 6.0 | 120 | 283 | 405 | 298 | 329 | 66.5 | 20.2 |
| 7.0 | 144 | 263 | 464 | 276 | 334 | 112 | 33.6 |
| 8.0 | 168 | 271 | 477 | 282 | 343 | 116 | 33.8 |
| 10 | 216 | 229 | 287 | 218 | 245 | 37.1 | 15.2 |
| 12 | 264 | 192 | 258 | 174 | 208 | 44.2 | 21.3 |
| 14 | 312 | 155 | 227 | 146 | 176 | 44.4 | 25.2 |
| 16 | 360 | 199 | 239 | 157 | 198 | 41.0 | 20.7 |
| 18 | 408 | 138 | 175 | 123 | 145 | 26.8 | 18.4 |
| 20 | 456 | 143 | 202 | 110 | 152 | 46.6 | 30.7 |
| 22 | 504 | 158 | 191 | 125 | 158 | 33.0 | 20.9 |
| 24 | 552 | 123 | 139 | 101 | 121 | 19.1 | 15.8 |
| 26 | 600 | 88.8 | 143 | 86.2 | 106 | 32.1 | 30.3 |
| 28 | 648 | 83.9 | 117 | 83.0 | 94.6 | 19.4 | 20.5 |
| 30 | 696 | 110 | 110 | 84.9 | 102 | 14.5 | 14.3 |
| 33 | 768 | 93.6 | 127 | 56.5 | 92.4 | 35.3 | 38.2 |
| 36 | 840 | 72.2 | 88.8 | 64.0 | 75.0 | 12.6 | 16.8 |
| 39 | 912 | 75.2 | 91.7 | 66.4 | 77.8 | 12.8 | 16.5 |
| 42 | 984 | 84.5 | 96.9 | 58.6 | 80.0 | 19.5 | 24.4 |
| 49 | 1152 | 31.5 | 32.6 | 24.2 | 29.4 | 4.57 | 15.5 |
| 56 | 1320 | 26.4 | 30.0 | 24.1 | 26.8 | 2.97 | 11.1 |
| 63 | 1488 | 25.3 | 22.2 | 18.6 | 22.0 | 3.35 | 15.2 |
| 70 | 1656 | 26.1 | 23.7 | 21.1 | 23.6 | 2.50 | 10.6 |
| 84 | 1992 | 20.4 | 19.5 | 17.8 | 19.2 | 1.32 | 6.86 |

### Further Example Formulations

Three further pharmaceutical compositions comprising an aqueous, injectable suspension have been formulated as tabulated below. All contained 3-chloro-7-(2-fluoro-4-(trifluoromethyl)phenyl)-2-methylbenzo[4,5] thieno[2,3-b]pyridin-4(1H)-one.

### Form 1 Polymorph

### Formulation 7: 3-chloro-7-(2-fluoro-4-(trifluoromethyl)phenyl)-2-methylbenzo[4,5] thieno[2,3-b]pyridin-4(1H)-one-Suspension, 300 mg/ml

| Component | % w/w | mg/ml |
|---|---|---|
| 3-chloro-7-(2-fluoro-4- | 30 | 300 |
| (trifluoromethyl)phenyl)-2- | | |
| methylbenzo[4,5] thieno[2,3- | | |
| b]pyridin-4(1H)-one | | |
| Poloxamer 338 | 4.5 | 45 |
| Polyethylene glycol 3350 | 3 | 30 |
| Mannitol | 2 | 20 |
| Water for injection | q.s. to 100 mL | q.s. 1 mL |

### Form 2 Polymorph

### Formulation 8: 3-chloro-7-(2-fluoro-4-(trifluoromethyl)phenyl)-2-methylbenzo[4,5] thieno[2,3-b]pyridin-4(1H)-one-Suspension, 300 mg/ml

| Component | % w/w | mg/ml |
|---|---|---|
| 3-chloro-7-(2-fluoro-4- | 30 | 300 |
| (trifluoromethyl)phenyl)-2- | | |
| methylbenzo[4,5] thieno[2,3- | | |
| b]pyridin-4(1H)-one | | |
| Poloxamer 338 | 4.5 | 45 |
| Sodium carboxymethyl cellulose | 0.66 | 6.6 |
| Mannitol | 3.5 | 35 |
| Water for injection | q.s. to 100 mL | q.s. 1 mL |

### Formulation 9: 3-chloro-7-(2-fluoro-4-(trifluoromethyl)phenyl)-2-methylbenzo[4,5] thieno[2,3-b]pyridin-4(1H)-one-Suspension, 300 mg/ml

Poloxamer 338, polysorbate 20, mannitol, 10 mM acetate buffer vehicle

| Component | % w/w | mg/ml |
|---|---|---|
| 3-chloro-7-(2-fluoro-4- | 30 | 300 |
| (trifluoromethyl)phenyl)-2- | | |
| methylbenzo[4,5] thieno[2,3- | | |
| b]pyridin-4(1H)-one | | |
| Poloxamer 338 | 4.5 | 45 |
| Polysorbate 20 | 1.5 | 15 |
| Sodium acetate trihydrate | 0.066 | 0.66 |
| Glacial acetic acid | 0.046 | 0.46 |
| Mannitol | 3.5 | 35 |
| Water for injection | q.s. to 100 mL | q.s. 1 mL |

1. A compound of Formula (I) 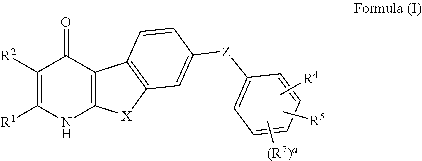
or a pharmaceutically acceptable salt thereof, wherein:
R¹ is C₁₋₅ alkyl, C₁₋₅ hydroxyalkyl or C₁-C₅ haloalkyl;
R² is hydrogen or halogen;
X is S, SO, or SO₂;
Z is a single bond, —O—, —C═C—, —C═C—CH₂—NR³—CH₂—, or —(CH₂)ₘ—, wherein m is 1, 2 or 3, and wherein R³ is hydrogen or a C₁₋₅ alkyl;
R⁴ is hydrogen, halogen, —CH₂F, —CHF₂, —CF₃, —OCH₂F, —OCHF₂, or —OCF₃;
R⁵ is —CF₃, —OCF₃, —(CH₂)_{q}—OH, wherein q is 1, 2, 3, or 4, or R⁵ is represented by the following group: 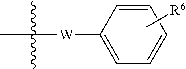
where [symbol] indicates a binding site to the phenyl ring and wherein W is —O—, or —CH₂—NR^{b}—Y—CH₂—, where R^{b} is hydrogen or C₁₋₅ alkyl, and Y is a single bond or Y is represented by the following group: 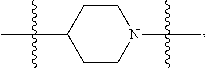 [SMILES: *C1CCN(*)CC1]
where [symbol] indicates a binding site to the amine and [symbol] indicates a binding site to the methylene group;
R⁶ is —CF₃, or —OCF₃;
R⁷ is halogen, —CHF₂, —CH₂F, or —CF₃; and
a is 0, 1, 2, or 3.

2. The compound or pharmaceutically acceptable salt thereof according to claim 1, wherein the compound is defined according to Formula Ia: 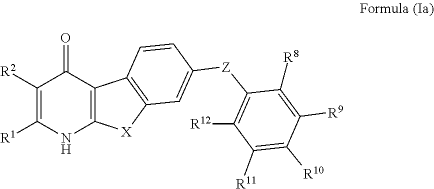
wherein R¹-R³, X, and Z are as defined in claim 1;
R⁸ is hydrogen, halogen, —CHF₂, —CH₂F, or —CF₃;
R⁹ is hydrogen, —CF₃, or —OCF₃;
R¹⁰ is hydrogen, —CF₃, —OCF₃, C₁-C₄ alkyl-OH, 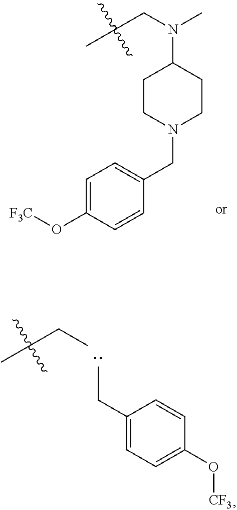
where [symbol] indicates a binding site to the phenyl ring;
R¹¹ is hydrogen, —CHF₂, —CH₂F, —CF₃;
R¹² is hydrogen, halogen, —CHF₂, —CH₂F, —CF₃, or —OCF₃.

3. The compound or pharmaceutically acceptable salt thereof according to claim 1, wherein R¹ is methyl; and/or wherein R² is chloro.

4. The compound or pharmaceutically acceptable salt thereof according to claim 1, wherein X is S.

5. The compound or pharmaceutically acceptable salt thereof according to any of claim 1, wherein Z is a single bond.

6. The compound or pharmaceutically acceptable salt thereof according to claim 1 selected from:
3-chloro-7-(2-fluoro-4-(trifluoromethyl)phenyl)-2-methylbenzo[4,5] thieno[2,3-b]pyridin-4(1H)-one;
sodium 3-chloro-7-[2-fluoro-4-(trifluoromethyl)phenyl]-2-methyl[1]benzothieno [2,3-b]pyridin-4-olate;
3-chloro-7-(2-fluoro-5-(trifluoromethyl)phenyl)-2-methylbenzo[4,5]thieno[2,3-b]pyridin-4(1H)-one;
3-chloro-2-methyl-7-(2-(trifluoromethoxy)phenyl)benzo[4,5]thieno[2,3-b]pyridin-4(1H)-one;
7-(2,4-bis(trifluoromethyl)phenyl)-3-chloro-2-methylbenzo[4,5]thieno[2,3-b]pyridin-4(1H)-one;
3-chloro-7-(4-(hydroxymethyl)phenyl)-2-methylbenzo[4,5]thieno[2,3-b]pyridin-4(1H)-one;
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one; or
3-chloro-2-methyl-7-(4-((methyl(1-(4-(trifluoromethoxy)benzyl)piperidin-4-yl)amino)methyl)phenyl)benzo[4,5]thieno[2,3-b]pyridin-4(1H)-one 2,2,2-trifluoroacetate.

7. The compound or pharmaceutically acceptable salt thereof according to claim 1, wherein the compound is:

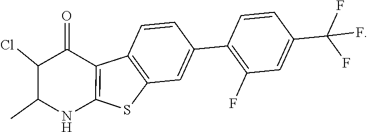 [SMILES: CC1Nc2sc3cc(-c4ccc(C(F)(F)F)cc4F)ccc3c2C(=O)C1Cl]

8. A crystalline form of 3-chloro-7-(2-fluoro-4-(trifluoromethyl)phenyl)-2-methylbenzo[4,5] thieno[2,3-b]pyridin-4(1H)-one characterised by an X-ray powder diffraction (XRPD) pattern comprising at least three diffraction angles, when measured using Cu K_{α} radiation, selected from the group consisting of either:
(i) about 5.2±0.1, 8.6±0.1, 10.5±0.1, 12.5±0.1, 13.5±0.1, 15.6±0.1, 18.8±0.1, 19.7±0.1, 20.9±0.1, 21.9±0.1, and 26.2±0.1 degrees 2θ; or
(ii) about 5.9±0.1, 9.9±0.1, 11.8±0.1, 13.9±0.1, 14.2±0.1, 15.4±0.1, 19.9±0.1, 23.2±0.1, 23.7±0.1, 24.2±0.1, 28.6±0.1, 29.7±0.1, 35.8±0.1 and 35.9±0.1 degrees 2θ.

9. The crystalline form of claim 8, wherein the crystalline form is characterised by an X-ray powder diffraction (XRPD) pattern substantially in accordance with FIG. 1 or FIG. 2.

10. The compound or pharmaceutically acceptable salt thereof according to of claim 1 for use in therapy.

11. A pharmaceutical composition comprising (a) the compound or pharmaceutically acceptable salt thereof according to claim 1; and (b) a pharmaceutically acceptable excipient or carrier.

12. The pharmaceutical composition according to claim 11, where the pharmaceutical composition is an injectable composition.

13. The compound or pharmaceutically acceptable salt thereof according to claim 1, for use in the treatment and/or prophylaxis of a parasitic protozoal infection.

14. The compound or pharmaceutically acceptable salt thereof according to claim 13, wherein the parasitic protozoal infection is malaria.

15. The compound or pharmaceutically acceptable salt thereof according to claim 13, wherein the parasitic protozoal infection is a Plasmodium falciparum infection.

16. A method of treating a parasitic protozoal infection in a human in need thereof, comprising administering to the human a therapeutically effective amount of the compound or pharmaceutically acceptable salt thereof according to claim 1.

17. The method according to claim 16, wherein the parasitic protozoal infection is malaria.

18. The method according to claim 16, wherein the parasitic protozoal infection is a Plasmodium falciparum infection.

19. (canceled)

20. (canceled)

21. (canceled)

22. A combination of (a) a compound or pharmaceutically acceptable salt thereof according to claim 1; and at least one other anti-malarial agent.

23. The combination according to claim 22, wherein the at least one other anti-malarial agent is atovaquone, 6-chloro-7-methoxy-2-methyl-3-{4-[4-(trifluoromethoxy)phenoxy]phenyl}quinolin-4(1H)-one, 6-chloro-7-methoxy-2-methyl-3-(4-(4-(trifluoromethoxy)phenoxy)phenyl)quinolin-4(1H)-one, a pharmaceutically salt thereof, or a combination thereof.

24. The crystalline form according to claim 8 for use in therapy.

25. A pharmaceutical composition comprising (a) the crystalline form of claim 8; and (b) a pharmaceutically acceptable excipient or carrier.

26. The crystalline form according to claim 8, for use in the treatment and/or prophylaxis of a parasitic protozoal infection.

27. The pharmaceutical composition according to claim 11, for use in the treatment and/or prophylaxis of a parasitic protozoal infection.

28. A method of treating a parasitic protozoal infection in a human in need thereof, comprising administering to the human a therapeutically effective amount of the crystalline form of claim 8.

29. A method of treating a parasitic protozoal infection in a human in need thereof, comprising administering to the human a therapeutically effective amount of the pharmaceutical composition according to claim 11.

30. A combination of (a) the crystalline form according to claim 8; and at least one other anti-malarial agent.
